(19) United States
(12) Patent Application Publication
(10) Pub. No.: US 2008/0015210 A1
(43) Pub. Date: Jan. 17, 2008

(54) Base-Stabilized Polyorthoester Formulations

(76) Inventors: Devang Shah, Redwood City, CA (US); John Barr, Redwood City, CA (US); Brian Baxter, Redwood City, CA (US); Jorge Heller, Ashland, OR (US)

Correspondence Address: HELLER EHRMAN LLP, 275 MIDDLEFIELD ROAD, MENLO PARK, CA, 94025-3506, US

(21) Appl. No.: 11/778,528

(22) Filed: Jul. 16, 2007

Related U.S. Application Data
(62) Division of application No. 11/433,834, filed on May 12, 2006.

(51) Int. Cl. A61K 47/30; A61K 31/4355; A61K 31/439; A61K 31/445

(52) U.S. Cl. 514/282; 514/299; 514/330; 514/772.7

(57) ABSTRACT

A stabilized semi-solid delivery vehicle contains a polyorthoester and an excipient, and a pharmaceutical composition contains an active agent, optionally a stabilizing agent, and the delivery vehicle. The pharmaceutical composition may be a topical, syringable, or injectable formulation; and is suitable for local delivery of the active agent. Methods of treatment are also disclosed.

### CROSS-REFERENCE TO RELATED APPLICATION

This application is a divisional of, and claims priority under 35 U.S.C. §120 to, U.S. application Ser. No. 11/433,834 filed May 12, 2006, the entire disclosure which is hereby incorporated by reference.

### BACKGROUND OF THE INVENTION

1. Field of the Invention

This invention relates to stabilized semi-solid delivery vehicles comprising a polyorthoester and an excipient, and to controlled release pharmaceutical compositions comprising the delivery vehicle, optionally a stabilizing agent, and an active agent. The pharmaceutical compositions may be in the form of a topical, syringable, or injectable formulation for local controlled delivery of the active agent.

2. Description of the Art

A large class of active agents such as antibiotics, antiseptics, corticosteroids, anti-neoplastics, and local anesthetics may be administered to the skin or mucous membrane by topical application, or by injection. The active agent may act locally or systemically. Topical delivery may be accomplished through the use of compositions such as ointments, creams, emulsions, solutions, suspensions and the like. Injections for delivery of the active agents include solutions, suspensions and emulsions. All of these preparations have been extensively used for delivery of active agents for years. However, these preparations suffer the disadvantage that they are short-acting and therefore they often have to be administered several times in a day to maintain a therapeutically effective dose level in the blood stream at the sites where the activity/treatment is required.

In recent years, a great deal of progress has been made to develop dosage forms which, after their administration, provide a long-term therapeutic response. These products may be achieved by microencapsulation, such as liposomes, microcapsules, microspheres, microparticles and the like. For this type of dosage forms, the active agents are typically entrapped or encapsulated in microcapsules, liposomes or microparticles which are then introduced into the body via injection or in the form of an implant. The release rate of the active agent from this type of dosage forms is controlled which eliminates the need for frequent dosing. However their manufacture is cumbersome which often results in high costs. In addition, they, in many cases, have low reproducibility and consequently lack of reliability in their release patterns. Furthermore, if an organic solvent is used in the manufacturing process, there could be organic solvent residues in the compositions which may be highly toxic. The use of an organic solvent is also undesirable for environmental and fire hazard reasons.

Interest in synthetic biodegradable polymers for the delivery of therapeutic agents began in the early 1970's with the work of Yolles et al., Polymer News, 1, 9-15 (1970) using poly(lactic acid). Since that time, numerous other polymers have been prepared and investigated as bioerodible matrices for the controlled release of active agents. U.S. Pat. Nos. 4,079,038, 4,093,709, 4,131,648, 4,138,344, 4,180,646, 4,304,767, 4,946,931 and 5,968,543 disclose various types of biodegradable or bioerodible polymers which may be used for controlled delivery of active agents. Many of these polymers may appear in the form of a semi-solid. However the semi-solid polymer materials are often too sticky. As a result, the active agents frequently cannot be easily and reliably released from the semi-solid polymer materials.

### SUMMARY OF THE INVENTION

One embodiment of the present invention provides a semi-solid delivery vehicle which comprises a polyorthoester and an excipient. The excipient is readily miscible with the polyorthoester and the resulting semi-solid delivery vehicle has a smooth and flowable texture. The polyorthoesters suitable for the invention are represented by formulae I, II, III and IV below.

Another embodiment of the present invention provides a controlled release semi-solid pharmaceutical composition for local controlled delivery of an active agent. The composition comprises an active agent and the semi-solid delivery vehicle.

Another embodiment of the present invention provides a semi-solid syringable or injectable composition for the controlled delivery of locally acting active agents, in particular local anesthetics.

In another embodiment, the above compositions comprising the polyorthoester can be homogeneously mixed with the excipient at room temperature without the use of a solvent. In another variation of the process, the polyorthoester can be homogeneously mixed with the excipient at between about 5 and 200° C., more preferably between about 20 and 150° C., and most preferably between about 25 and 100° C. In one variation, the polyorthoester can be at one temperature, for example at about 70° C., and the excipient can be at a different temperature, for example at about 120° C., and the two components are mixed to attain a final temperature that is above room temperature. The desired temperatures for each of the two components will be based on the type of the polyorthoester and the excipient selected. The resulting semi-solid delivery vehicle and controlled-release pharmaceutical compositions have a useful texture and viscosity, and the release rate of the active agent from the compositions can also be conveniently and reliably adjusted to accommodate the desired therapeutic effect.

Thus, in one aspect, this invention provides a semi-solid delivery vehicle, comprising:

(i) a polyorthoester of formula I, formula II, formula III or formula IV

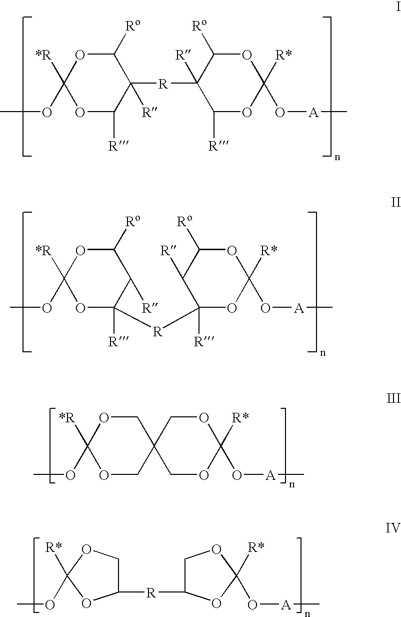

where:

R is a bond, —(CH₂)ₐ—, or —(CH₂)_{b}—O—(CH₂)_{c}—; where a is an integer of 1 to 10, and b and c are independently integers of 1 to 5;

R* is a C₁₋₄ alkyl;

R^{o}, R″ and R′″ are each independently H or C₁₋₄ alkyl;

n is an integer of at least 5; and

A is R¹, R³, or R⁴, where

R¹ is:

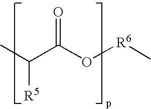

where:

p is an integer of 1 to 20;

R³ and R⁶ are each independently:

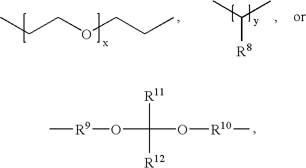

where:

x is an integer of 0 to 30;

y is an integer of 2 to 200;

R⁸ is hydrogen or C₁₋₄ alkyl;

R⁹ and R¹⁰ are independently C₁₋₁₂ alkylene;

R¹¹ is hydrogen or C₁₋₆ alkyl and R¹² is C₁₋₆ alkyl; or R¹¹ and R¹² together are C₃₋₁₀ alkylene;

R⁴ is a diol containing at least one functional group independently selected from amide, imide, urea, and urethane groups; and

R⁵ is hydrogen or C₁₋₄ alkyl; and

in which at least 0.01 mol percent of the A units are of the formula R¹, and wherein the polyorthoester has a lifetime of 12 hours or less in vitro.

In another aspect, this invention provides a controlled release semi-solid pharmaceutical composition comprising:

(a) a basic active agent; and

(b) as a delivery vehicle, the semi-solid delivery vehicle described above.

In another aspect, this invention provides a controlled release semi-solid pharmaceutical composition comprising:

(a) an active agent;

(b) a stabilizing agent; and

(c) as a delivery vehicle, the semi-solid delivery vehicle described above.

In another aspect, this invention provides a method of treating a disease state treatable by controlled release local administration of an active agent, in particular treating pain by administration of a local anesthetic, comprising locally administering a therapeutically effective amount of the active agent in the form of the pharmaceutical composition described above.

In another aspect, this invention provides a method of treating a disease state treatable by controlled release local administration of an active agent, in particular treating or preventing of nausea and/or emesis by administration of an antiemetic agent, comprising locally administering a therapeutically effective amount of the active agent in the form of the pharmaceutical composition described above.

### DETAILED DESCRIPTION OF THE INVENTION

### DEFINITIONS

Unless defined otherwise in this specification, all technical and scientific terms are used herein according to their conventional definitions as they are commonly used and understood by those of ordinary skill in the art of synthetic chemistry, pharmacology and medicine.

“Active agent” includes any compound or mixture of compounds which produces a beneficial or useful result. Active agents are distinguishable from such components as vehicles, carriers, diluents, lubricants, binders and other formulating aids, and encapsulating or otherwise protective components. Examples of active agents and their pharmaceutically acceptable salts, are pharmaceutical, agricultural or cosmetic agents. Suitable pharmaceutical agents include locally or systemically acting pharmaceutically active agents which may be administered to a subject by topical or intralesional application (including, for example, applying to abraded skin, lacerations, puncture wounds, etc., as well as into surgical incisions) or by injection, such as subcutaneous, intradermal, intramuscular, intraocular, or intra-articular injection. Examples of these agents include, but not limited to, anti-infectives (including antibiotics, antivirals, fungicides, scabicides or pediculicides), antiseptics (e.g., benzalkonium chloride, benzethonium chloride, chlorhexidine gluconate, mafenide acetate, methylbenzethonium chloride, nitrofurazone, nitromersol and the like), steroids (e.g., estrogens, progestins, androgens, adrenocorticoids, and the like), therapeutic polypeptides (e.g. insulin, crythropoietin, morphogenic proteins such as bone morphogenic protein, and the like), analgesics and anti-inflammatory agents (e.g., ketorolac, COX-1 inhibitors, COX-2 inhibitors, and the like), cancer chemotherapeutic agents (e.g., mechlorethamine, cyclophosphamide, fluorouracil, thioguanine, carmustine, lomustine, melphalan, chlorambucil, streptozocin, methotrexate, vincristine, bleomycin, vinblastine, vindesine, dactinomycin, daunorubicin, doxorubicin, tamoxifen, and the like), narcotics (e.g., morphine, meperidine, codeine, and the like), local anesthetics (e.g., the amide- or anilide-type local anesthetics such as bupivacaine, dibucaine, mepivacaine, procaine, lidocaine, tetracaine, and the like), antiemetic agents such as ondansetron, granisetron, tropisetron, metoclopramide, domperidone, scopolamine, and the like, antiangiogenic agents (e.g., combrestatin, contortrostatin, anti-VEGF, and the like), polysaccharides, vaccines, antigens, DNA and other polynucleotides, antisense oligonucleotides, and the like. The present invention may also be applied to other locally acting active agents, such as astringents, antiperspirants, irritants, rubefacients, vesicants, sclerosing agents, caustics, escharotics, keratolytic agents, sunscreens and a variety of dermatologics including hypopigmenting and antipruritic agents. The term “active agents” further includes biocides such as fungicides, pesticides, and herbicides, plant growth promoters or inhibitors, preservatives, disinfectants, air purifiers and nutrients. Pro-drugs of the active agents are included within the scope of the present invention.

“Basic active agent” means an active agent as defined above wherein the active agent has basic properties or functionalities such as basic active agents comprising compounds that are Lewis bases having nonbonding pairs of electrons or Bronsted bases. Examples of such as agents include those having an amine or nitrogen containing group. The basic active agent may also include compositions comprising an active agent that has basic properties or finctionalities as defined above.

“Biologically active organic compound” means an active agent, as defined above, wherein the active agent is an organic compound.

“Alkyl” denotes a linear saturated hydrocarbyl having from one to the number of carbon atoms designated, or a branched or cyclic saturated hydrocarbyl having from three to the number of carbon atoms designated (e.g., C₁₋₄ alkyl). Examples of alkyl include methyl, ethyl, n-propyl, isopropyl, cyclopropyl, n-butyl, t-butyl, cyclopropylmethyl, and the like.

“Alkylene” denotes a straight or branched chain divalent, trivalent or tetravalent alkylene radical having from one to the number of carbon atoms designated, or a branched or cyclic saturated cycloalkylenyl having from three to the number of carbon atoms designated (e.g., C₁₋₄ alkylenyl, or C₃₋₇ cycloalkylenyl), and include, for example 1,2-ethylene, 1,3-propylene, 1,2-propylene, 1,4-butylene, 1,5-pentylene, 1,6-hexylene, 1,2,5-hexylene, 1,3,6-hexylene, 1,7-heptylene, and the like.

“Bioerodible” and “bioerodibility” refer to the degradation, disassembly or digestion of the polyorthoester by action of a biological environment, including the action of living organisms and most notably at physiological pH and temperature. A principal mechanism for bioerosion of the polyorthoesters of the present invention is hydrolysis of linkages between and within the units of the polyorthoester.

“Comprising” is an inclusive term interpreted to mean containing, embracing, covering or including the elements listed following the term, but not excluding other unrecited elements.

“Controlled release”, “sustained release”, and similar terms are used to denote a mode of active agent delivery that occurs when the active agent is released from the delivery vehicle at an ascertainable and controllable rate over a period of time, rather than dispersed immediately upon application or injection. Controlled or sustained release may extend for hours, days or months, and may vary as a function of numerous factors. For the pharmaceutical composition of the present invention, the rate of release will depend on the type of the excipient selected and the concentration of the excipient in the composition. Another determinant of the rate of release is the rate of hydrolysis of the linkages between and within the units of the polyorthoester. The rate of hydrolysis in turn may be controlled by the composition of the polyorthoester and the number of hydrolyzable bonds in the polyorthoester. Other factors determining the rate of release of an active agent from the present pharmaceutical composition include particle size, solubility of the active agent, acidity of the medium (either internal or external to the matrix) and physical and chemical properties of the active agent in the matrix.

“Delivery vehicle” denotes a composition which has the flictions including transporting an active agent to a site of interest, controlling the rate of access to, or release of, the active agent by sequestration or other means, and facilitating the application of the agent to the region where its activity is needed.

“Matrix” denotes the physical structure of the polyorthoester or delivery vehicle which essentially retains the active agent in a manner preventing release of the agent until the polyorthoester erodes or decomposes.

“Polyorthoester-compatible” refers to the properties of an excipient which, when mixed with the polyorthoester, forms a single phase and does not cause any physical or chemical changes to the polyorthoester.

“Pro-drug” denotes a pharmacologically inactive or less active form of a compound which must be changed or metabolized in vivo, e.g., by biological fluids or enzymes, by a subject after administration into a pharmacologically active or more active form of the compound in order to produce the desired pharmacological effect. Prodrugs of a compound can be prepared by modifying one or more functional group(s) present in the compound in such a way that the modification(s) may be cleaved in vivo to release the parent compound. Prodrugs include compounds wherein a hydroxy, amino, sulfhydryl, carboxy or carbonyl group in a compound is bonded to any group that can be cleaved in vivo to regenerate the free hydroxyl, amino, sulflhydryl, carboxy or carbonyl group respectively. Examples of prodrugs include, but are not limited to, esters (e.g. acetate, dialkylaminoacetates, formates, phosphates, sulfates and benzoate derivatives) and carbamates of hydroxy functional groups (e.g. N,N-dimethylcarbonyl), esters of carboxyl functional groups (e.g. ethyl esters, morpholinoethanol esters), N-acyl derivatives (e.g. N-acetyl), N-Mannich bases, Schiff bases and enaminones of amino functional groups, oximes, acetals, ketals, and enol esters of ketones and aldehyde functional groups in a compound, and the like.

“Semi-solid” denotes the mechano-physical state of a material that is flowable under moderate stress. More specifically, the semi-solid material should have a viscosity between about 10,000 and 3,000,000 cps, especially between about 30,000 and 500,000 cps. Preferably the formulation is easily syringable or injectable, meaning that it can readily be dispensed from a conventional tube of the kind well known for topical or ophthalmic formulations, from a needleless syringe, or from a syringe with a 16 gauge or smaller needle, such as 16-25 gauge.

“Sequestration” is the confinement or retention of an active agent within the internal spaces of a polyorthoester matrix. Sequestration of an active agent within the matrix may limit the toxic effect of the agent, prolong the time of action of the agent in a controlled manner, permit the release of the agent in a precisely defined location in an organism, or protect unstable agents against the action of the environment.

“Stabilizing agent” means an organic or inorganic agent, or mixture of such agents, that when provided in a sufficient amount, extends the useful lifetime of the polymer in an aqueous environment, or in vivo, with respect to the lifetime of an unstabilized polymer. As used herein, the stabilizing agent may be an agent that is added to a polymer containing an active agent, or wherein the active agent itself is the stabilizing agent.

A “therapeutically effective amount” means the amount that, when administered to an animal for treating a disease, is sufficient to effect treatment for that disease.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease). For the purposes of this invention, a “disease” includes pain.

A “unit” denotes an individual segment of a polyorthoester chain, which consists of the residue of a diketene acetal molecule and the residue of a polyol.

An “α-hydroxy acid containing” unit denotes a unit where A is R¹, i.e. in which the polyol is prepared from an α-hydroxy acid or cyclic diester thereof and a diol of the formula HO—R⁵—OH. The fraction of the polyorthoester that is α-hydroxy acid containing units affects the rate of hydrolysis (or bioerodibility) of the polyorthoester, and in turn, the release rate of the active agent.

Polyorthoesters

The polyorthoesters are of formula I, II, III or IV: 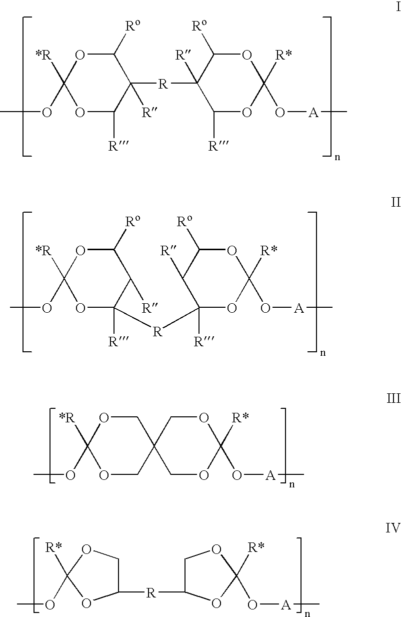
in which at least 0.01 mol % of the A units are of the formula R¹.

The structure of the polyorthoester useful for the present invention, as shown in formula I, II, III or IV, is one of alternating residues of a diketene acetal and a diol, with each adjacent pair of diketene acetal residues being separated by the residue of one polyol, preferably a diol.

In the presence of water, the α-hydroxy acid containing units are readily hydrolyzed at a body temperature of 37° C. and a physiological pH, to produce the corresponding hydroxyacids. These hydroxyacids then act as acidic catalysts to control the hydrolysis rate of the polyorthoester without the addition of exogenous acid. When the polyorthoester is used as a delivery vehicle or matrix entrapping an active agent, the hydrolysis of the polyorthoester causes release of the active agent.

Polyorthoesters having a higher mole percentage of the “α-hydroxy acid containing” units will have a higher rate of bioerodibility. Preferred polyorthoesters are those in which the mole percentage of the “α-hydroxy acid containing” units is at least 0.01 mole percent, in the range of about 0.01 to about 50 mole percent, more preferably from about 0.05 to about 30 mole percent, for example from about 0.1 to about 25 mole percent, especially from about 1 to about 20 mole percent. The mole percentage of the “α-hydroxy acid containing” units appropriate to achieve the desired composition will vary from formulation to formulation.

The starting polyorthoesters of the present invention that do not comprise an active agent and/or a stabilizing agent are limited to the polyorthoesters that have a lifetime of about 30 minutes to about 12 hours.

Preferred polyorthoesters are those where:

n is an integer of 5 to 1000, or n is an integer of 5 to 500;

the polyorthoester has a molecular weight of 1000 to 20,000, preferably 1000 to 10,000, more preferably 1000 to 8000;

R⁵ is hydrogen or methyl;

R⁶ is:

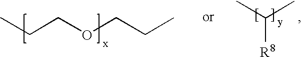

where x is an integer of 0 to 10, especially 1 to 4; y is an integer of 2 to 30, especially 2 to 10; and R⁸ is hydrogen or methyl;

R³ is:

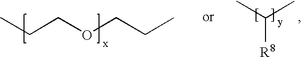

where x is an integer of 0 to 10, especially 1 to 4; y is an integer of 2 to 30, especially 2 to 10; and R⁸ is hydrogen or methyl;

R⁴ is selected from the residue of an aliphatic diol of 2 to 20 carbon atoms, preferably 2 to 10 carbon atoms, interrupted by one or two amide, imide, urea, or urethane groups;

the proportion of units in which A is R¹ is about 0.01-50 mol %, preferably 0.05-30 mol %, more preferably 0.1-25 mol %; and

the proportion of units in which A is R⁴ is less than 20%, preferably less than 10%, especially less than 5%.

While the presence of any of these preferences results in a polyorthoester that is more preferred than the same polyorthoester in which the preference is not met, the preferences are generally independent, and polyorthoesters in which a greater number of preferences is met will generally result in a polyorthoester that is more preferred than that in which a lesser number of preferences is met.

Preparation of the Polyorthoesters

The polyorthoesters are prepared according to the methods described in U.S. Pat. Nos. 4,549,010 and 5,968,543. Specifically, the polyorthoesters are prepared by the reaction of a diketene acetal of formula V or formula VI: 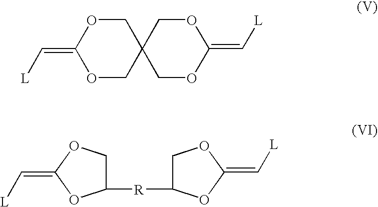
where L is hydrogen or a C₁₋₃ alkyl, with a diol of the formula HO—R¹—OH and at least one diol of the formulae HO—R³—OH or HO—R⁴—OH.

To form the polyorthoester using a mixture of the two types of the diols, the mixture is formed with selected proportions based on the desired characteristics of the polyorthoester. The use of increasing amounts of diols in which A is R¹ increases the bioerodibility of the polyorthoester, and the use of such diols in which R⁶ is a polyethyleneoxide moiety or an alkane increases the softness of the polymer; and the use of diols in which A is R³ and/or R⁶ increases the softness of the polyorthoester, especially when these diols are low molecular weight polyethylene glycols or aliphatic diols. The use of diols in which A is R⁴ also generally increases the hardness of the polyorthoester because of the hydrogen bonding between adjacent chains of the polyorthoester, and may or may not be desirable depending on the other diols used.

The preparation of the diketene acetals of the types of formula V and formula VI is disclosed in U.S. Pat. Nos. 4,304,767, 4,532,335, and 5,968,543; and will be known to a person of ordinary skill in the art. A typical method is the condensation of a bis(diol) of formula VII (i.e. pentacrythritol) or formula VIII: 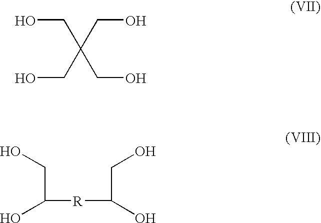
with two equivalents of a 2-halocarboxaldehyde dialkyl acetal, such as 2-bromoacetaldehyde diethyl acetal, followed by dehydrohalogenation to give the diketene acetal. The condensation of a glycol with diethylbromoacetals is described in Roberts et al., J. Am. Chew. Soc., 80, 1247-1254 (1958), and dehydrohalogenation is described in Beyerstedt et al., J. Am. Chem. Soc., 58, 529-553 (1936).

The diketene acetals may also be prepared by the isomerization of divinyl acetals. Thus, for example, 3,9-di(ethylidene)-2,4,8,10-tetraoxaspiro[5.5]undecane (DETOSU) may be prepared by the isomerization of 3,9-divinyl-2,4,8,10-tetraoxaspiro[5.5]undecane, using n-butyllithium in ethylenediamine. The isomerization of the double bond is described in Corey et al., J. Org. Chem., 38, 3224 (1973). The divinyl acetals may be prepared by the condensation of the bis(diol) of formula VII or formula VIII with two equivalents of a vinylic aldehyde, such as acrolein or crotonaldehyde, or their dialkyl acetals, such as acrolein dimethyl acetal, and such condensation reactions are well known.

The bis(diol) of formula VII where R is a bond is erythritol. The bis(diol) of formula VIII where R is —(CH₂)ₐ— may be prepared by the oxidation of an α,ω-diene, such as 1,3-butadiene or 1,5-hexadiene, with an oxidizing reagent such as osmium tetroxide/hydrogen peroxide, or by other methods known in the art, to give the bis(diol). In one variation, the bis(diol) of formulae VII and VIII may be further optionally substituted. The bis(diol) of formula VIII where R is —(CH₂)_{b}—O—(CH₂)_{c}— may be prepared by the reaction of an ω-hydroxy-α-ol such as allyl alcohol, with an ω-haloalkyloxirane, such as epichlorohydrin, to form an ω-epoxy-α-olefin with the backbone interrupted by an oxygen atom, such as 2-allyloxymethyloxirane, which is then oxidized with an oxidizing reagent such as osmium tetroxide/hydrogen peroxide, or by other methods known in the art, to give the bis(diol).

The diols of the formulae HO—R¹—OH, HO—R³—OH, and HO—R⁴—OH are prepared according to methods known in the art, and as described, for example, in U.S. Pat. Nos. 4,549,010 and 5,968,543. Some of the diols are commercially available. The diol of the formula HO—R¹—OH that comprises a polyester moiety may be prepared by reacting a diol of the formula HO—R³—OH or HO—R⁶—OH with between 0.5 and 10 molar equivalents of a cyclic diester of an α-hydroxy acid, such as lactide or glycolide, and allowing the reaction to proceed at 100-200° C. for about 12 hours to about 48 hours. Although particular solvents are not required for this reaction, organic solvents such as dimethylacetamide, dimethyl sulfoxide, dimethylformamide, acetonitrile, pyrrolidone, tetrahydrofuran, and methylbutyl ether may be used.

The preparation of diols, in particular the diol of the formula HO—R³—OH is generally disclosed in Heller et al, J. Polymer Sci., Polymer Letters Ed. 18:293-297 (1980), by reacting an appropriate divinyl ether with an excess of an appropriate diol. Diols of the formula HO—R⁴—OH include diols where R⁴ is R′CONR″R′ (amide), R′CONR″COR′ (imide), R′NR″CONR″R′ (urea), and R′OCONR″R′ (urethane), where each R′ is independently an aliphatic, aromatic, or aromatic/aliphatic straight or branched chain hydrocarbyl, especially a straight or branched chain alkyl of 2 to 22 carbon atoms, especially 2 to 10 carbon atoms, and more especially 2 to 5 carbon atoms, and R″ is hydrogen or C₁₋₆ alkyl, especially hydrogen or methyl, more especially hydrogen.

Some representative diols of the formula HO—R⁴—OH include N,N′-bis-(2-hydroxyethyl)terephthalamide, N,N′-bis-(2-hydroxyethyl)pyromellitic diimide, 1,1′-methylenedip-phenylene)bis-[3-(2-hydroxyethyl)urea], N,N′-bis-(2-hydroxyethyl)oxamide, 1,3-bis(2-hydroxyethyl)urea, 3-hydroxy-N-(2-hydroxyethyl)propionamide, 4-hydroxy-N-(3-hydroxypropyl)butyramide, and bis(2-hydroxyethyl)ethylenedicarbamate. These diols are known to the art in reported syntheses and many are commercially available. Representative diols of the formula HO—(CH₂)ₙ—NHCO—(CH₂)ₘ—OH where n is an integer of 2 to 6 and m is an integer of 2 to 5 are made by the reaction of 2-aminoethanol, 3-aminopropanol, 4-aminobutanol, 5-aminopentanol, or 6-aminohexanol with β-propiolactone, γ-butyrolactone, δ-valerolactone, or ε-caprolactone. Representative diols of the formula HO—(CH₂)ₙ—NHCOO—(CH₂)ₘ—OH where n and m are each integers of 2 to 6 are made by the reaction of the same aminoalcohols just mentioned with cyclic carbonates of the formula 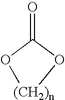
such as ethylene carbonate. Bis-amide diols of the formula HO-A-NHCO—B—CONE-A-OH are prepared by the reaction of a diacid, optionally in activated form, such as the diacyldihalide, with two equivalents of a hydroxy-amine. Other methods of preparation of the diols of the formula HO—R⁴—OH are known in the art.

Once made, the diol of the formula HO—R¹—OH and the diolks) of the formulae HO—R³—OH, HO—R⁶—OH and HO—R⁴—OH in the desired proportions are mixed with the diketene acetal of formula V or formula VI, in a slightly less than 1;1 (e.g. 0.5:1-0.9:1) ratio of total number of moles of diketene acetal to total number of moles of diols, in a suitable solvent at ambient temperature. The condensation reaction between the diketene acetal and the diols is carried out under conditions which are described in, for example, U.S. Pat. Nos. 4,304,767, 4,549,010, and 5,968,543, and are well known to those skilled in the art; and will also be readily apparent from the structures of the reactants themselves. Suitable solvents are aprotic solvents, such as dimethylacetamide, dimethyl sulfoxide, dimethylformamide, acetonitrile, acetone, ethyl acetate, pyrrolidone, tetrahydrofuran, and methylbutyl ether, and the like. Catalysts are not required for this reaction, but when used, suitable catalysts are iodine in pyridine, p-toluenesulfonic acid; salicylic acid, Lewis acids (such as boron trichloride, boron trifluoride, boron trichloride etherate, boron trifluoride etherate, stannic oxychloride, phosphorous oxychloride, zinc chloride, phosphorus pentachloride, antimony pentafluoride, stannous octoate, stannic chloride, diethyl zinc, and mixtures thereof); and Bronsted catalysts (such as polyphosphoric acid, crosslinked polystyrene sulfonic acid, acidic silica gel, and mixtures thereof). A typical amount of catalyst used is about 0.2% by weight relative to the diketene acetal. Smaller or larger amounts can also be used, such as 0.005% to about 2.0% by weight relative to the diketene acetal. Once the reaction is complete, the reaction mixture is allowed to cool and concentrated by rotoevaporation under vacuum. The concentrated mixture may be further dried under vacuum at an elevated temperature.

The polyorthoesters may also be prepared by reaction of the diketene acetal with the chosen diol(s) under similar reaction conditions, but in the presence of a “chain stopper” (a reagent that terminates polyorthoester chain formation). Suitable chain stoppers are C₅₋₂₀ alkanols, especially C₁₀₋₂₀ alkanols. The chain stopper is preferably present in from 1-20 mol % based on the diketene acetal. The polyorthoesters thus prepared have low molecular weights with a lower molecular weight dispersion than those prepared by the reaction of the diketene acetals with only diols, and are therefore especially suitable for this invention.

Preferably, the polyethylene glycol copolymers have a molecular weight between 1,000 and 8,000, more preferably, between 3,000 and 7,000, and most preferably about 5,000 to 6,000.

The Excipients:

The excipients suitable for the present invention are pharmaceutically acceptable and polyorthoester-compatible materials. They are liquid at room temperature, and are readily miscible with the polyorthoesters.

Suitable excipients include poly(ethylene glycol) ether derivatives having a molecular weight of between 200 and 4,000, such as poly(ethylene glycol) mono- or di-alkyl ethers, preferably poly(ethylene glycol)monomethyl ether 550 or poly(ethylene glycol)dimethyl ether 250; poly(ethylene glycol)copolymers having a molecular weight of between 200 and 10,000 such as poly(ethylene glycol-co-polypropylene glycol); propylene glycol mono- or di-esters of a C₂₋₁₉ aliphatic carboxylic acid or a mixture of such acids, such as propylene glycol dicaprylate or dicaprate; mono-, di- or tri-glycerides of a C₂₋₁₉ aliphatic carboxylic acid or a mixture of such acids, such as glyceryl caprylate, glyceryl caprate, glyceryl caprylate/caprate, glyceryl caprylate/caprate/laurate, glycofurol and similar ethoxylated tetrahydrofurfuryl alcohols and their C₁₋₄ alkyl ethers and C₂₋₁₉ aliphatic carboxylic acid esters; and biocompatible oils such as sunflower oil, sesame oil and other non- or partially-hydrogenated vegetable oils.

Most of these materials are commercially available, for example, from Aldrich Chemical Company (Milwaukee, Wis.) and from Abitec Corporation (Columbus, Ohio), LIPO Chemicals Inc. (Paterson, N.J.), and Jarchem Industries, Inc. (Newark, N.J.).

The Delivery Vehicle:

The delivery vehicle comprises a polyorthoester and an excipient selected from those described in preceding sections.

The concentrations of the polyorthoester and the excipient in the delivery vehicle may vary. For example, the concentration of the excipient in the vehicle may be in the range of 1-99% by weight, preferably 5-80% weight, especially 20-60% by weight of the vehicle.

While the singular form is used to describe the polyorthoester and excipient in this application, it is understood that more than one polyorthoesters and excipients selected from the groups described above may be used in the delivery vehicle.

The delivery vehicle is prepared by mixing or blending together the polyorthoester and the excipient. The mixing or blending can be performed by any methods at a temperature less than about 50° C., e.g. at room temperature, in the absence of solvents, using any suitable devices to achieve a homogeneous, flowable and non-tacky semi-solid blend at room temperature. In another aspect of the invention, the mixing or blending can be performed by any methods at a temperature of about between 5 to 200° C., more preferably about between 20 to 150° C., and more preferably about between 25 and 100° C., depending on the nature of the starting material selected, as noted above, to achieve a homogeneous, flowable and tacky or non-tacky semi-solid blend at room temperature.

Semi-Solid Pharmaceutical Compositions:

If the basic active agent is itself a liquid or semi-solid, it may be mixed with the delivery vehicle in the same manner as the delivery vehicle was formed, i.e. conventional blending of semi-solid formulations. Such blending is carried out in a manner suitable to obtain a homogeneous distribution of the components throughout the formulation, by mixing the components in any order necessary to achieve such homogeneity. However, the basic active agent is typically a solid. It is desirable that the particle size of the basic active agent be sufficiently small (for example, 1-100 μm, especially 5-50 μm) so that the resulting composition is smooth. Therefore, unless the basic active agent is already in micron-sized powder form, it is generally first milled into fine particles preferably less than 100 μm and sieved before mixing with the other ingredients. The mechanical mixing process is performed at room temperature, preferably under vacuum in order to avoid air bubbles. In another aspect of the process, the mechanical mixing process may be performed at room temperature or above room temperature without the use of any vacuum. If desired, further size reduction of the size of the particles of the basic active agent can be carried out by passing the semi-solid mixture through a ball mill or roller mill to achieve a homogeneous and uniform pharmaceutical composition.

The basic active agent may be mixed with the delivery vehicle already formed or directly mixed together with the polyorthoester and the excipient. In another aspect of the invention, the basic active agent, delivery vehicle, polyorthoester and excipient may be mixed together in any suitable order to obtain the product with the desired characteristics.

The basic active agent is present in the composition in an amount which is effective to provide a desired biological or therapeutic effect. Because of the sustained release nature of the compositions, the basic active agent usually is present in an amount which is greater than the conventional single dose. The concentration of the basic active agent in the semi-solid polyorthoester composition can vary over a wide range (e.g., 0.1-80 wt. %, preferably 0.3-60 wt. %, more preferably 0.5-40 wt. %, such as 1-30 wt. %, based on the composition as a whole) depending on a variety of factors, such as the release profile of the composition, the therapeutically effective dose of the active agent, and the desired length of the time period during which the active agent is released. In one aspect of the invention, the concentration of the basic active agent in the semi-solid polyorthoester composition is between about 1-5 wt. %, more preferably between about 2-3 wt. %.

The concentration of the polyorthoester may be 1-99 wt. %, preferably 5-40 wt. %, of the composition. The total concentration of the excipient is 1-90 wt. %, preferably 5-60 wt. %, more preferably 10-50 wt. %, of the composition.

The polyorthoester containing a basic active agent may further comprise a stabilizing agent. In one aspect, the stabilizing agent is an inorganic or organic compound or complex.

The semi-solid pharmaceutical composition of the present invention of formula I, II, III or IV may be employed for the delivery of biologically active agents and maintain a drug concentration in the blood within the therapeutic range for about 10 hours or less. Certain strategies have been used to obtain more stabilized controlled release formulations, including changing the particular nature of the functional groups or monomeric units within the polymer to afford polymers that are useful as carriers or matrixes for drugs. However, these polymers do not incorporate latent acid units and have molecular weights as high as 200,000. See U.S. Pat. No. 4,304,767.

The semi-solid pharmaceutical compositions of the present invention have significantly improved stability characteristics in vitro. In one aspect, the compositions comprise a latent acid and have lower molecular weights of about 8,000 or less.

It is also understood that while not required, other pharmaceutically acceptable inert agents such as coloring agents and preservatives may also be incorporated into the composition.

The semi-solid pharmaceutical composition of the present invention has an improved texture which is non-tacky and flowable. In another aspect of the invention, the semi-solid pharmaceutical composition of the present invention has an improved texture which is tacky and also flowable. As used herein, the term “tacky” refers to a physical property of the composition in which the composition is sticky when lightly touched. The composition therefore can be conveniently applied to the skin or mucous membrane in the manner of a convention al cream or gel. Preferably the formulation is easily syringable or injectable, meaning that it can readily be dispensed from a conventional tube of the kind well known for topical or ophthalmic formulations, from a needleless syringe, or from a syringe with a 16 gauge or smaller needle (such as 16-25 gauge), and injected subcutaneously, intradermally or intramuscularly. The formulation may be applied using various methods known in the art, including by syringe, injectable or tube dispenser, for example, directly or indirectly to the skin or a wound.

After topical application, administration by injection, or any other routes of administration, including surface or subcutaneous application to open wounds, the active agent is released from the composition in a sustained and controlled manner. The rate of release may be regulated or controlled in a variety of ways to accommodate the desired therapeutic effect. The rate may be increased or decreased by altering the mole percentage of the α-hydroxy acid containing units in the polyorthoester, or by selecting a particular excipient, or by altering the amount of the selected excipient, or the combination thereof.

The compositions are also stable. The release rates of the active agent are not affected by irradiation for sterilization.

Particular Compositions and Their Uses:

Exemplary compositions of this invention, and their uses, include:

(1) compositions containing local anesthetics, optionally in combination with glucocorticosteroids such as dexamethasone, cortisone, hydrocortisone, prednisone, prednisolone, beclomethasone, betamethasone, flunisolide, fluocinolone acetonide, fluocinonide, triamcinolone, including deposition of the compositions into surgical sites, and the like, for the prolonged relief of local pain or a prolonged nerve blockade. This use is discussed further below;

(2) compositions containing cancer chemotherapeutic agents, such as those listed above under “Active Agents”, for deposition by syringe or by injection into tumors or operative sites from which a tumor has been ablated, for tumor control or treatment and/or the suppression of regrowth of the tumor from residual tumor cells after ablation of the tumor;

(3) compositions containing progestogens, such as flurogestone, medroxyprogesterone, norgestrel, norgestimate, norethindrone, and the like, for estrus synchronization or contraception;

(4) compositions containing antimetabolites such as fluorouracil and the like, as an adjunct to glaucoma filtering surgery; compositions containing antiangiogenic agents such as combrestatin, for the treatment of macular degeneration and retinal angiogenesis; and other compositions for the controlled release of ophthalmic drugs to the eye;

(5) compositions containing therapeutic polypeptides proteins), such as insulin, LHRH antagonists, and the like, for the controlled delivery of these polypeptides, avoiding the need for daily or other frequent injection;

(6) compositions containing anti-inflammatory agents such as the NSAIDs, e.g. ibuprofen, naproxen, COX-1 or COX-2 inhibitors, and the like, or glucocorticosteroids, for intra-articular application or injection;

(7) compositions containing antibiotics, for the prevention or treatment of infection, especially for deposition into surgical sites to suppress post-operative infection, or into or on wounds, for the suppression of infection (e.g. from foreign bodies in the wound);

(8) compositions containing morphogenic proteins such as bone morphogenic protein;

(9) compositions containing DNA or other polynucleotides, such as antisense oligonucleotides;

(10) compositions containing antiemetic agents;

(11) compositions containing antigens in vaccines; and

(12) compositions comprising a combination of two or more of the above active agents for concurrent therapeutic applications.

In another aspect, the composition comprises an inorganic salt or a mixture of inorganic salts. Inorganic salts that may be employed are those that stabilize the composition and extend the lifetime of the composition in vivo. In one aspect, the inorganic salts are basic inorganic salts. The inorganic salts may comprise metal oxides or metal hydroxides, such as sodium hydroxide, lithium hydroxide, calcium hydroxide, and potassium hydroxides. In another aspect, the inorganic salt consists of magnesium hydroxide.

In another aspect, the salts are organometallic salts such as sodium methoxide, sodium ethoxide, lithium methoxide, lithium ethoxide, magnesium methoxide, and the like. Delivery of Controlled-release Antiemetic Agents

The present invention further relates to a method for the treatment or prevention of emesis in a patient which comprises administering an 5-HT₃ antagonist, wherein the 5-HT₃ antagonist minimize the side effects of nausea and/or emesis associated with other pharmacological agents.

In a further aspect of the present invention, there is provided a pharmaceutical composition for the treatment or prevention of emesis comprising an HT₃ antagonist, together with at least one pharmaceutically acceptable carrier or excipient.

As used herein, the term “emesis” includes nausea and vomiting. The HT₃ antagonists in the semi-solid injectable form of the present invention are beneficial in the therapy of acute, delayed or anticipatory emesis, including emesis induced by chemotherapy, radiation, toxins, viral or bacterial infections, pregnancy, vestibular disorders (e.g. motion sickness, vertigo, dizziness and Meniere's disease), surgery, migraine, and variations in intracranial pressure. The HT₃ antagonists of use in the invention are of particular benefit in the therapy of emesis induced by radiation and/or by chemotherapy, for example during the treatment of cancer, or radiation sickness; and in the treatment of post-operative nausea and vomiting. The HT₃ antagonists in the semi-solid injectable form of the invention are beneficial in the therapy of emesis induced by antineoplastic (cytotoxic) agents including those routinely used in cancer chemotherapy, and emesis induced by other pharmacological agents, for example, alpha-2 adrenoceptor antagonists, such as yohimbine, MK-912 and MK-467, and type IV cyclic nucleotide phosphodiesterase (PDE4) inhibitors, such as RS 14203, CT-2450 and rolipram.

Particular examples of chemotherapeutic agents are described, for example, by D. J. Stewart in Nausea and Vomiting: Recent Research and Clinical Advances, ed. J. Kucharczyk et al., CRC Press Inc., Boca Raton, Fla., USA, 1991, pages 177-203, see page 188. Examples of commonly used chemotherapeutic agents include cisplatin, dacarbazine (DTIC), dactinomycin, mechlorethamine (nitrogen mustard), streptozocin, cyclophosphamnide, carmustine (BCN-U), lomustine (CCNU), doxorubicin (adriamycin), daunorubicin, procarbazine, mitomycin, cytarabine, etoposide, methotrexate, 5-fluorouracil, vinblastine, vincristine, bleomycin and chlorambucil (see R. J. Gralle et al. in Cancer Treatment Reports, 1984, 68, 163-172).

Many of the antiemetic agents are conventionally used in the form of their acid addition salts, as this provides solubility in aqueous injection media. However, because the presence of the large amount of acid within such a local antiemetic acid addition salt will result in more rapid degradation of the polyorthoesters and rapid release of the antiemetic agent, it is generally desirable to use the antiemetic agent in the free base form. Alternatively, the antiemetic may be used with only a small proportion of the acid addition salt present (addition of small quantities of the acid addition salt may provide enhanced release if desired).

The semi-solid injectable form of an antiemetic agent of the present invention is prepared by incorporating the antiemetic agent into the delivery vehicle in a manner as described above. The concentration of the antiemetic agent may vary from about 0.1-80 wt. %, preferably from about 0.2-60 wt. %, more preferably from about 0.5-40 wt. %, most preferably from about 1-5 wt. %, for example, about 2-3 wt. %. The semi-solid composition is then filled into a syringe with a 16-25 gauge needle, and injected into sites that have been determined to be most effective. The semi-solid injectable composition of the present invention can be used for controlled delivery of both slightly soluble and soluble antiemetic agents.

Suitable classes of antiemetic agents employed in the present invention include, for example, a 5-HT₃ antagonist such as ondansetron, granisetron or tropisetron; a dopamine antagonist such as metoclopramide or domperidone; an anticholinergic agent such as scopolamine; a GABA_{B} receptor agonist such as baclofen; an NK₁ receptor antagonist as described, for example, in WO 97/49710; or a GABA_{A}α₂ and/or α₃ receptor agonist as described in WO 99/67245.

The 5-HT₃ antagonists employed in the present invention are also useful for the treatment or prevention of emesis in conjunction with the use of other antiemetic agents known in the art.

In one particular aspect, suitable classes of other antiemetic agents of use in conjunction with the present invention include, for example, alpha-2 adrenoreceptor agonists including for example, clonidine, apraclonidine, para-aminoclonidine, brimonidine, naphazoline, oxymetazoline, tetrahydrozoline, tramazoline, detomidine, medetomidine, dexmedetomidine, B-HT 920, B-HIT 933, xylazine, rilmenidine, guanabenz, guanfacine, labetatol, phenylephrine, mephentermine, metaraminol, methoxamine and xylazine.

As noted, the compounds or agents employed in the present invention are also useful for the treatment or prevention of emesis in conjunction with another antiemetic agents known in the art, such as a 5-HT₃ antagonist, a dopamine antagonist, an anticholinergic agent, a GABA_{B} receptor agonist, an NK₁ receptor antagonist, and a GABA_{A}α₂ and/or α₃ receptor agonist.

In another aspect of the invention, the antiemetic agents as a single agent or as a combination, may be used independently in the form of a salt or salts or mixtures of the agent and the salt of the agent. Suitable pharmaceutically acceptable salts of the compounds of use in the present invention include acid addition salts which may, for example, be formed by mixing a solution of the compound with a solution of a pharmaceutically acceptable non-toxic acid such as hydrochloric acid, iodic acid, fumaric acid, maleic acid, succinic acid, acetic acid, citric acid, tartaric acid, carbonic acid, phosphoric acid, sulfuric acid and the like. Salts of amine groups may also comprise the quaternary ammonium salts in which the amino nitrogen atom carries an alkyl, alkenyl, alkynyl or aralkyl group. Where the compound carries an acidic group, for example a carboxylic acid group, the present invention also contemplates salts thereof, preferably non-toxic pharmaceutically acceptable salts thereof, such as the sodium, potassium and calcium salts thereof.

It will be appreciated that when using a combination of the present invention, the 5-HT₃ antagonists and the other antiemetic agent will be administered to a patient together in the in the semi-solid injectable form of the invention. In one aspect of the invention, the compounds may be in the same pharmaceutically acceptable carrier and therefore administered simultaneously.

When administered in combination, either as a single product in the semi-solid injectable form or as separate pharmaceutical compositions, the 5-HT₃ antagonists and the other antiemetic medicament are to be presented in a ratio which is consistent with the manifestation of the desired effect. In particular, the ratio by weight of the 5-HT₃ antagonists and the other antiemetic agent will suitably be between 0.001 to 1 and 1000 to 1, and especially between 0.01 to 1 and 100 to 1.

The present invention is further directed to a method for ameliorating the symptoms attendant to emesis in a patient comprising administering to the patient a 5-HT₃ antagonist. In accordance with the present invention the 5-HT₃ antagonists is administered to a patient in a quantity sufficient to treat or prevent the symptoms and/or underlying etiology associated with emesis in the patient.

Delivery of Controlled-Release Local Anesthetics

Local anesthetics induce a temporary nerve conduction block and provide pain relief which lasts from a few minutes to a few hours. They are frequently used to prevent pain in surgical procedures, dental manipulations or injuries.

The synthetic local anesthetics may be divided into two groups: the slightly soluble compounds and the soluble compounds. Conventionally, the soluble local anesthetics can be applied topically and by injection, and the slightly soluble local anesthetics are used only for surface application. The local anesthetics conventionally administered by injection can also be divided into two groups, esters and non-esters. The esters include (1) benzoic acid esters (piperocaine, meprylcaine and isobucaine); (2) para-aminobenzoic acid esters (procaine, tetracaine, butethamine, propoxycaine, chloroprocaine); (3) meta-aminobenzoic acid esters (metabutethamine, primacaine); and (4) para-ethoxybenzoic acid ester (parethoxycaine). The non-esters are anilides (amides or nonesters) which include bupivacaine, lidocaine, mepivacaine, pyrrocaine and prilocaine.

Many of the local anesthetics are conventionally used in the form of their acid addition salts, as this provides solubility in aqueous injection media. However, because the presence of the large amount of acid within such a local anesthetic acid addition salt will result in more rapid degradation of the polyorthoesters and release of the local anesthetic, it is generally desirable to use the local anesthetics in free base form, or with only a small proportion of the acid addition salt present (addition of small quantities of the acid addition salt may provide enhanced release if desired).

The semi-solid injectable form of a local anesthetic of the present invention is prepared by incorporating the local anesthetic into the delivery vehicle in a manner as described above. The concentration of the local anesthetic may vary from about 0.1-80 wt. %, preferably from about 1-60 wt. %, more preferably from about 0.5-40 wt. %, most preferably from about 1-5 wt. %, for example, about 2-3 wt. %. The semi-solid composition can be administered directly into surgical incision sites or subcutaneously via a suitable sized needle. In another aspect, the semi-solid composition is then filled into a syringe with a 16-25 gauge needle, and injected into sites that are painful or to be subjected to surgical procedures. The semi-solid injectable composition of the present invention can be used for controlled delivery of both slightly soluble and soluble local anesthetics.

Because the duration of action of a local anesthetic is proportional to the time during which it is in actual contact with nervous tissues, the present injectable delivery system can maintain localization of the anesthetic at the nerve for an extended period of time which will greatly prolong the effect of the anesthetic.

A number of authors, including Berde et al., U.S. Pat. No. 6,046,187 and related patents, have suggested that the co-administration of a glucocorticosteroid may prolong or otherwise enhance the effect of local anesthetics, especially controlled-release local anesthetics; and formulations containing a local anesthetic and a glucocorticosteroid, and their uses for controlled release local anesthesia, are within the scope of this invention.

### ASPECTS OF THE INVENTION

In one aspect of the invention, there is provided a pharmaceutical composition comprising;

(A) a semi-solid delivery vehicle, comprising
a polyorthoester of formula I,

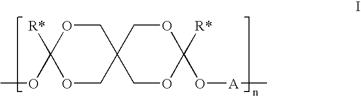

where:

R* is a C₁₋₄ alkyl;

n is an integer of at least 5; and

A is R¹, R³, or R⁴, where

R¹ is:

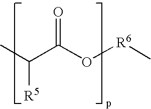

where:

p is an integer of 1 to 20;

R³ and R⁶ are each independently:

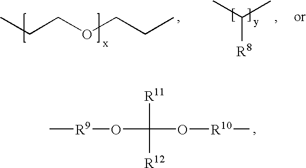

where:

x is an integer of 0 to 30;

y is an integer of 2 to 200;

R⁸ is hydrogen or C₁₋₄ alkyl;

R⁹ and R¹⁰ are independently C₁₋₁₂ alkylene;

R¹¹ is hydrogen or C₁₋₆ alkyl and R¹² is C₁₋₆ alkyl; or R¹¹ and R¹² together are C₃₋₁₀ alkylene;

R⁴ is a diol containing at least one functional group independently selected from amide, imide, urea, and urethane groups; and

R⁵ is hydrogen or C₁₋₄ alkyl;

in which at least 0.01 mol percent of the A units are of the formula R¹, and wherein the polyorthoester has a lifetime of 12 hours or less in vitro;

(B) a pharmaceutically acceptable, polyorthoester-compatible liquid excipient selected from polyethylene glycol ether derivatives having a molecular weight between 200 and 4000, polyethylene glycol copolymers having a molecular weight between 200 and 10,000, mono-, di- or tri-glycerides of a C₂₋₁₉ aliphatic carboxylic acid or a mixture of such acids, alkoxylated tetrahydrofurfuryl alcohols and their C₁₋₄ alkyl ethers and C₂₋₁₉ aliphatic carboxylic acid esters, and biocompatible oils; and

(C) a polyorthoester stabilizing agent that extends the lifetime of the polyorthoester by at least one lifetime, wherein the stabilizing agent is a basic biologically active agent.

Examples of polyethylene glycol (PEG) ethers include PEG dimethyl ethers, with molecular weights of 200 to 4,000 and preferably with a molecular weight of 500 to 2,000. In one variation, the PEG ethers are the PEG dipropyl ethers and the PEG dibutyl ethers. In one aspect of the invention, the PEG copolymers are polyethylene glycol polymers having at least one different monomer unit in the polymer.

In one variation, there is provided the above pharmaceutical composition, wherein the polyorthoester has a lifetime of 6 hours or less in vitro. As used herein, the lifetime of the polymer is defined as the amount of time it takes for the initial polymer to decompose or disintegrate. Such lifetime may be measured by or correlated with the amount of a particular drug or excipient in the polymer to be released from the polymer. In another variation, there is provided the above pharmaceutical composition, wherein the polyorthoester has a lifetime of 4 hours, or alternately, 2 hours or less in vitro.

In one aspect of the invention, the stabilizing agent extends the lifetime of the polyorthoester by at least two lifetimes, at least three lifetimes, at least four lifetimes, or at least five lifetimes. In another aspect, the stabilizing agent extends the lifetime of the polyorthoester by at least ten lifetimes. In yet another aspect, the stabilizing agent extends the lifetime of the polyorthoester by at least thirty lifetimes.

In general, the stabilized POE polymers comprises from about 0.01 to 30%, in particular from about 0.05 to 7%, preferably from about 0.1 to 5%, especially from about 1 to 4% of the stabilizing agent. In another variation of the above pharmaceutical composition, the stabilizing agent is an amine comprising biologically active organic compound or its salt. In another variation, the biologically active organic compound is selected from the group consisting of anti-infectives, antiseptics, steroids, therapeutic polypeptides, anti-inflammatory agents, cancer chemotherapeutic agents, narcotics, local anesthetics, antiemetics, antiangiogenic agents, vaccines, antigens, DNA, RNA and antisense oligonucleotides. In yet another variation, the active agent is a therapeutic polypeptide.

In another variation, the active agent is a local anesthetic. In one aspect, the concentration of the anesthetic agent in the composition is about 1-5 wt. %. In one aspect, the local anesthetic is selected from the group consisting of bupivacaine, lidocaine, mepivacaine, pyrrocaine and prilocaine. In one variation, the pharmaceutical composition further comprises a glucocorticosteroid. In another aspect, the active agent is an antiangiogenic agent. In one variation of the above composition, the active agent is a cancer chemotherapeutic agent. In another variation, the active agent is an antibiotic. In another variation, the active agent is an anti-inflammatory agent.

In yet another variation, the active agent is an antiemetic agent. In one aspect, the fraction of the antiemetic agent is from 0.1% to 80% by weight of the composition. In another aspect, the fraction of the antiemetic agent is from 0.1% to 5% by weight of the composition. In another aspect, the fraction of the antiemetic agent is from 1% to 5% by weight of the composition. In one variation, the composition is in topical, syringable, or injectable form.

In one aspect, the invention provides any one of the above pharmaceutical composition where the antiemetic agent is selected from the group consisting of 5-HT₃ antagonists, a dopamine antagonists, an anticholinergic agents, a GABA_{B} receptor agonists, an NK₁ receptor antagonists, and a GABA_{A}α₂ and/or α₃ receptor agonists. In another aspect, the antiemetic agent is a 5-HT₃ antagonist. In yet another aspect, the 5-HT₃ antagonist is selected from the group consisting of ondansetron, granisetron and tropisetron. In another aspect, the antiemetic agent further comprises a second antiemetic agent to form a combination composition. In one variation, the second antiemetic agent is selected from the group consisting of alpha-2 adrenoreceptor agonists, a dopamine antagonist, an anticholinergic agent, a GABA_{B} receptor agonist, an NK₁ receptor antagonist, and a GABA_{A}α₂ and/or α₃ receptor agonist.

In another variation, the alpha-2 adrenoreceptor agonists is selected from the group consisting of clonidine, apraclonidine, para-aminoclonidine, brimonidine, naphazoline, oxymetazoline, tetrahydrozoline, tramazoline, detomidine, medetomidine, dexmedetomidine, B-HT 920, B-HIT 933, xylazine, rilmenidine, guanabenz, guanfacine, labetalol, phenylephrine, mephentermine, metaraminol, methoxamine and xylazine.

In one aspect, the invention provides a method for the treatment of emesis induced by a chemotherapeutic agent, by radiation-induced nausea and vomiting, and/or by post operative induced nausea and vomiting in a patient in need thereof which comprises administering to the patient the composition comprising the 5-HT₃ antagonist described above. In one variation, there is provided a method wherein the 5-HT₃ antagonist is selected from the group consisting of ondansetron, granisetron and tropisetron. In another variation, the patient is a human. In one aspect, the administration comprises the deposition of the composition comprising the 5-HT₃ antagonist into a surgical site.

In one aspect of the invention, there is provided a method for the prevention of emesis induced by a chemotherapeutic agent in a patient in need thereof which comprises administering to the patient the composition comprising the 5-HT₃ antagonist described above. In one variation, the 5-HT₃ antagonist is selected from the group consisting of ondansetron, granisetron and tropisetron. In one aspect, the invention provides a method for ameliorating the symptoms attendant to emesis induced by a chemotherapeutic agent, by radiation-induced nausea and vomiting, and/or by post operative induced nausea and vomiting in a patient comprising administering to the patient in need thereof the composition comprising the 5-HT₃ antagonist described above. In another aspect, the 5-HT₃ antagonist is selected from the group consisting of ondansetron, granisetron and tropisetron. In each of the above aspect and variation, the patient is a human.

In another aspect, the invention provides a method for the prevention of emesis induced by a chemotherapeutic agent, by radiation-induced nausea and vomiting, and/or by post operative induced nausea and vomiting in a patient in need thereof which comprises administering to the patient the composition comprising the 5-HT₃ antagonist described above, and a second antiemetic agent. In one variation, the second antiemetic agent is a compound selected from the group consisting of alpha-2 adrenoreceptor agonists, a dopaamine antagonist, an anticholinergic agent, a GABA_{B} receptor agonist, an NK₁ receptor antagonist, and a GABA_{A}α₂ and/or α₃ receptor agonist. In one variation of the above composition, the antiemetic agent is selected from the group consisting of ondansetron, granisetron and tropisetron. In one variation of the above, the fraction of the antiemetic agent is from 0.1% to 5% by weight of the composition.

In one aspect of the invention, there is provided a polyorthoester of formula I or formula II:

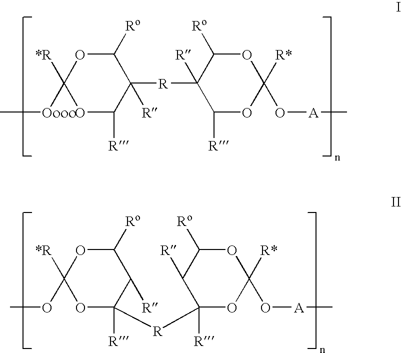

where:

R is a bond, —(CH₂)ₐ—, or —(CH₂)_{b}—O—(CH₂)_{c}—; where a is an integer of 1 to 10, and b and c are independently integers of 1 to 5;

R* is a C₁₋₄ alkyl;

R^{o}, R″ and R′″ are each independently H or C₁₋₄ alkyl, provided that at least one of R^{o} or R′″ is C₁₋₄ alkyl in formula I;

n is an integer of at least 5; and

A is R¹, R³, or R⁴, where

R¹ is:

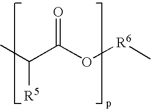

where:

p is an integer of 1 to 20;

R³ and R⁶ are each independently:

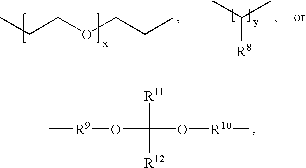

where:

x is an integer of 0 to 30;

y is an integer of 2 to 200;

R⁸ is hydrogen or C₁₋₄ alkyl;

R⁹ and R¹⁰ are independently C₁₋₁₂ alkylene;

R¹⁰ is hydrogen or C₁₋₆ alkyl and R¹² is C₁₋₆ alkyl; or R¹¹ and R¹² together are C₃₋₁₀ alkylene;

R⁴ is a diol containing at least one functional group independently selected from amide, imide, urea, and urethane groups; and

R⁵ is hydrogen or C₁₋₄ alkyl;

in which at least 0.01 mol percent of the A units are of the formula R¹.

In one variation of the above composition, n is 5 to 500. In another variation of the above, R is —CH₂OCH₂—. In another variation, R* is ethyl. In yet another variation, R⁰, R″ or R′″ are each independently methyl or ethyl. In a particular variation of the above, the polyorthoester comprises at least 0.1 mol % of units in which A is R¹. In one variation, the above polyorthoester comprises about 0.5-50 mol % of units in which A is R¹. In another variation, the polyorthoester comprises about 1-30 mol % of units in which A is R¹.

In a particular variation of the above polyorthoester, p is 1 to 2. In another variation, R⁵ is hydrogen. In yet another variation, R⁵ is methyl. In a particular variation of the above, R⁶ is —CH₂CH₂OCH₂CH₂OCH₂CH₂—. In another particular variation of the above polyorthoester, HO—R³—OH is triethylene glycol or 1,10-decariediol.

In one aspect, the invention provides a process for preparing a polyorthoester of formula I or formula II:

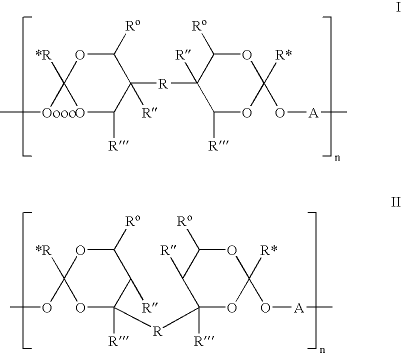

where:

R is a bond, —(CH₂)ₐ—, or —(CH₂)_{b}—O—(CH₂)_{c}—; where a is an integer of 1 to 10, and b and c are independently integers of 1 to 5;

R* is a C₁₋₄ alkyl;

R^{o}, R″ and R′″ are each independently H or C₁₋₄ alkyl, provided that at least one of R^{o} or R′″ is C₁₋₄ alkyl in formula I;

n is an integer of at least 5; and

A is R¹, R³, or R⁴, where

R¹ is:

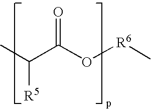

where:

p is an integer of 1 to 20;

R³ and R⁶ are each independently:

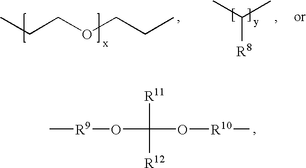

where:

x is an integer of 0 to 30;

y is an integer of 2 to 200;

R⁸ is hydrogen or C₁₋₄ alkyl;

R⁹ and R¹⁰ are independently C₁₋₁₂ alkylene;

R¹¹ is hydrogen or C₁₋₆ alkyl and R¹² is C₁₋₆ alkyl; or R¹¹ and R¹² together are C₃₋₁₀ alkylene;

R⁴ is a diol containing at least one functional group independently selected from amide, imide, urea, and urethane groups; and

R⁵ is hydrogen or C₁₋₄ alkyl;

in which at least 0.01 mol percent of the A units are of the formula R¹; and

R⁴ is (i) the residue of a diol containing at least one amine functionality incorporated therein, or

(ii) the residue of a diol containing at least one functional group independently selected from amide, imide, urea, and urethane groups,

the process comprising reacting a di(ketene acetal) of formula Ia or formula IIa:

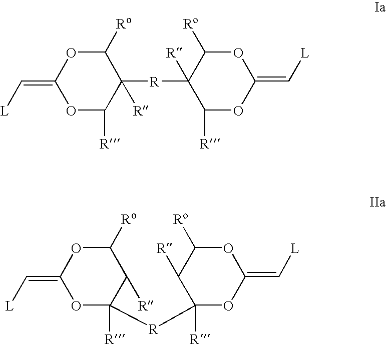

where L is hydrogen or a C₁₋₃ alkyl;

with a diol of the formula HO—R¹—OH, HO—R³—OH, or HO—R⁴—OH, or a mixture thereof.

In another aspect of the invention there is provided a polyorthoester that is the product of a reaction between:

(a) a di(ketene acetal) of formula Ia or formula IIa:

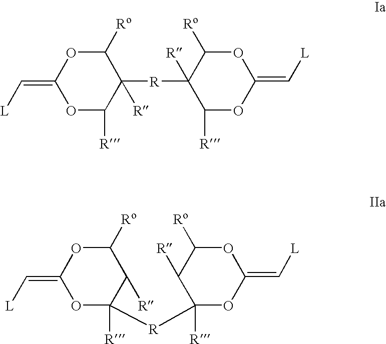

where:

R is a bond, —(CH₂)ₐ—, or —(CH₂)_{b}—O—(CH₂)_{c}—; where a is an integer of 1 to 10, and b and c are independently integers of 1 to 5;

R^{o}, R″ and R′″ are each independently H or C₁₋₄ alkyl, provided that at least one of R^{o} or R′″ is C₁₋₄ alkyl in formula Ia;

L is hydrogen or a C₁-C₃ alkyl; and

(b) a polyol or mixture of polyols.

In one variation of the above, at least one of the polyols is a polyol having more than two hydroxy functional groups. In one aspect of the invention, there is provided a device for orthopedic restoration or tissue regeneration comprising a polyorthoester of the above aspects and variations. In yet another aspect of the invention, there is provided a method of stabilizing a polyorthoester polymer having a lifetime of 12 hours or less in vitro, the method comprising of adding to the polymer stabilizing agent wherein the stabilizing agent is a basic biologically active agent and the stabilizing agent extends the lifetime of the polymer by at least one lifetime or at least two lifetimes.

In one variation of the above method, the polymer hydrolyzes with a lifetime of about 12 hours or less in vitro in the absence of the stabilizing agent, and the stabilizing agent extends the lifetime of the polymer by at least one lifetime. In another variation of the method, the polymer hydrolyzes with a lifetime of about 6 hours or less in vitro in the absence of the stabilizing agent, and the stabilizing agent extends the lifetime of the polymer by at least two lifetimes. In yet another variation, the stabilizing agent is a basic agent and/or a basic biologically active agent.

In a particular method of the above, the stabilizing agent is an amine comprising biologically active organic compound or its salt. In one variation, of the above method, the biologically active organic compound is selected from the group consisting of anti-infectives, antiseptics, steroids, therapeutic polypeptides, anti-inflammatory agents, cancer chemotherapeutic agents, narcotics, local anesthetics, antiemetics, antiangiogenic agents, vaccines, antigens, DNA, RNA and antisense oligonucleotides. In another variation, the active compound is a local anesthetic. In a particular variation of the above method, the local anesthetic is selected from the group consisting of bupivacaine, lidocaine, mepivacaine, pyrrocaine and prilocaine.

In a particular variation, the composition of the above method further comprises a glucocorticosteroid. In a particular variation of the above method, the active organic compound is an antiemetic agent. In one variation, the antiemetic agent is selected from the group consisting of ondansetron, granisetron and tropisetron. In one particular variation of the above method, the fraction of the antiemetic agent is from 0.1% to 5% by weight of the composition.

In one variation of the above method, the stabilizing agent is an inorganic salt, organic salts of alkali metals or alkaline earth metals, or mixtures thereof In another variation, the stabilizing agent is a biologically active organic compound and an inorganic salt, organic salt of alkali metal or an alkaline earth metal, or mixtures thereof In another variation, the inorganic salt is not magnesium hydroxide, calcium carbonate, sodium acetate, or hydroxyapatite.

In another aspect, the invention provides a pharmaceutical composition comprising a polyorthoester polymer sensitive to hydrolysis in vitro, wherein the polymer has a lifetime of 12 hours or less in vitro, and a stabilizing agent. In one variation of the above pharmaceutical composition, the stabilizing agent is a basic agent and/or a basic biologically active agent. In one particular variation, the basic biologically active agent is an amine comprising biologically active organic compound or its salt. In a particular variation of the above, the fraction of the active agent is from 1% to 10% by weight of the composition.

In one aspect, the fraction of the active agent is from 1% to 60% by weight of the composition. In another aspect, the fraction of the active agent is from 5% to 30%.

In another aspect of the invention, there is provided a method of treating a disease state treatable by controlled release local administration of an active agent, comprising locally administering a therapeutically effective amount of the active agent in the form of a pharmaceutical composition of any one of the above embodiments, aspects and variation of the invention.

In another aspect of the invention, there is provided a method of preventing or relieving local pain at a site in a mammal, comprising administering to the site a therapeutically effective amount of a local anesthetic in the form of a pharmaceutically acceptable composition of any of the above embodiments, aspects and variations. In another aspect, there is provided a process for the preparation of the delivery vehicle of the above embodiment, comprising mixing the components (A) and (B) in the absence of a solvent, at a temperature between about 20 and 150° C.

In yet another aspect, there is provided a process for the preparation of the pharmaceutical composition of the above embodiment, wherein the active agent is in solid form, comprising:

(1) optionally milling the active agent to reduce the particle size of the active agent;

(2) mixing the active agent, the stabilizing agent, and the delivery vehicle; and

(3) optionally milling the composition to reduce the particle size of the active agent. In yet another aspect, there is provided a process for the preparation of the pharmaceutical composition of the above embodiment, where the active agent and the stabilizing agent is in solid form, comprising:

(1) warming the polyorthoester to 70° C.;

(2) dissolving the active agent and the stabilizing agent in the excipient at 120-150° C.; and

(3) mixing the 70° C. polyorthoester into the 120° C. solution of the active agent and stabilizing agent in the excipient with an agitator under the following conditions to obtain a homogeneous distribution of the components:

(a) under an inert atmosphere;

(b) optionally warming the mixing vessel to 70° C.; or

(c) optionally allowing the temperature of the mixture to equilibrate under ambient conditions during the mixing process.

In another aspect of the invention, there is provided the semi-solid delivery vehicle above where the concentration of the polyoroester ranges from 1% to 99% by weight. In one variation, the polyorthoester has a molecular weight between 1,000 and 20,000. In another aspect, the fraction of the A units that are of the formula R¹ is between 1 and 90 mol percent.

In one aspect of the invention, the polyorthoester is of formula I, where none of the units have A equal to R², R³ is: 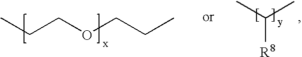
where x is an integer of 0 to 10; y is an integer of 2 to 30; and R⁶ is: 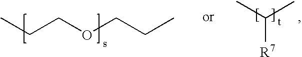
where s is an integer of 0 to 10, t is an integer of 2 to 30, and R⁵, R⁷, and R⁸ are independently hydrogen or methyl. In one variation, R³ and R⁶ are both —(CH₂—CH₂—O)₂—(CH₂—CH₂)—, R⁵ is methyl, and p is 1 or 2. In another variation, R³ and R⁶ are both —(CH₂—CH₂—O)₉—(CH₂—CH₂)—, R⁵ is methyl, and p is 1 or 2.

In one aspect of the invention, there is provided a pharmaceutical composition of wherein the anesthetic agent is selected from the group consisting of bupivacaine, lidocaine, mepivacaine, pyrrocaine and prilocaine. In one variation, the concentration of the anesthetic agent in the composition is about 1-5 wt. %. In one aspect of the invention, there is provided the above composition wherein the antiemetic agent is granisetron. In one variation, the fraction of the antiemetic agent is from 0. 1% to 80% by weight of the composition. In another variation, the fraction of the antiemetic agent is from 1% to 5% by weight of the composition. In another aspect of the invention, the composition is in topical, syringable, or injectable form.

In yet another aspect of the invention, there is provided a composition wherein the antiemetic agent is selected from the group consisting of 5-HT₃ antagonists, dopamine antagonists, anticholinergic agents, GABA_{B} receptor agonists, NK₁ receptor antagonists, and GABA_{A}α₂ and/or α₃ receptor agonists. In one variation, the antiemetic agent is a 5-HT₃ antagonist. In another variation, the 5-HT₃ antagonist is selected from the group consisting of ondansetron, granisetron and tropisetron.

In yet another aspect, there is provided the above pharmaceutical composition further comprising a second antiemetic agent to form a combination composition. In one variation, the second antiemetic agent is selected from the group consisting of alpha-2 adrenoreceptor agonists, a dopamine antagonist, an anticholinergic agent, a GABA_{B} receptor agonist, an NK₁ receptor antagonist, and a GABA_{A}α₂ and/or α₃ receptor agonist. In another variation, the alpha-2 adrenoreceptor agonists is selected from the group consisting of clonidine, apraclonidine, para-aminoclonidine, brimonidine, naphazoline, oxymetazoline, tetrahydrozoline, tramazoline, detomidine, medetomidine, dexmedetomidine, B-HT 920, B-HIT 933, xylazine, rilmenidine, guanabenz, guanfacine, labetalol, phenylephrine, mephentermine, metaraminol, methoxamine and xylazine.

In another aspect of the invention, there is provided a method for the treatment of emesis induced by a chemotherapeutic agent, by radiation-induced nausea and vomiting, and/or by post operative induced nausea and vomiting in a patient in need thereof which comprises administering to the patient the above composition comprising the 5-HT₃ antagonist of the invention. In one variation of the above method, the 5-HT₃ antagonist is selected from the group consisting of ondansetron, granisetron and tropisetron. In another variation of the above method, the patient is a human. In yet another variation of the method, the administration comprises the deposition of the 5-HT₃ antagonist into a surgical site.

In another aspect of the invention, there is provided a method for the prevention of emesis induced by a chemotherapeutic agent in a patient in need thereof which comprises administering to the patient the above composition comprising the 5-HT₃ antagonist. In one variation, the 5-HT₃ antagonist is selected from the group consisting of ondansetron, granisetron and tropisetron. In another variation of the above method, the patient is a human.

In another aspect, there is provided a method for ameliorating the symptoms attendant to emesis induced by a chemotherapeutic agent, by radiation-induced nausea and vomiting, and/or by post operative induced nausea and vomiting in a patient comprising administering to the patient in need thereof a composition of the invention comprising an 5-HT₃ antagonist. In one variation, the 5-HT₃ antagonist is selected from the group consisting of ondansetron, granisetron and tropisetron. In one variation of the above method, the patient is a human.

In another aspect of the invention, there is provided a method for the prevention of emesis induced by a chemotherapeutic agent, by radiation-induced nausea and vomiting, and/or by post operative induced nausea and vomiting in a patient in need thereof which comprises administering to the patient a composition of the invention comprising a 5-HT₃ antagonist, and a second antiemetic agent. In one variation, the second antiemetic agent is a compound selected from the group consisting of alpha-2 adrenoreceptor agonists, a dopamine antagonist, an anticholinergic agent, a GABA_{B} receptor agonist, an NK₁ receptor antagonist, and a GABA_{A}α₂ and/or α₃ receptor agonist.

In yet another aspect of the invention, there is provided a process for the preparation of the delivery vehicle of the present invention, comprising mixing the components (A), (B) and (C) in the absence of a solvent, at a temperature between about 20 and 150° C.

In yet another aspect, there is provided a process for the preparation of the pharmaceutical composition above where the antiemetic agent is in solid form, comprising: (1) optionally milling the active agent to reduce the particle size of the active agent; (2) mixing the active agent and the delivery vehicle; and (3) optionally milling the composition to reduce the particle size of the active agent.

In yet another aspect, there is provided a process for the preparation of the pharmaceutical composition of the present invention where the antiemetic agent and/or the anesthetic agent is in solid form, comprising; (1) warming the polyorthoester to 70° C.; (2) dissolving the active agent in the excipient at 120-150° C.; and (3) mixing the 70° C. polyorthoester into the 120° C. solution of the active agent in the excipient with an agitator under the following conditions to obtain a homogeneous distribution of the components: (a) under an inert atmosphere, such as an argon or nitrogen atmosphere (b) optionally warming the mixing vessel to 70° C.; or (c) optionally allowing the temperature of the mixture to equilibrate under ambient conditions during the mixing process.

In yet another aspect, there is provided a process for the preparation of the pharmaceutical composition of the present invention where the antiemetic agent and/or the anesthetic agent is in solid form, comprising: (1) warming the polyorthoester to 70° C.; (2) dissolving the active agent in the excipient at 120-150° C.; and (3) mixing the 70° C. polyorthoester into the 120° C. solution of the active agent in the excipient with an agitator under the following conditions to obtain a homogeneous distribution of the components: (a) under an inert atmosphere, such as an argon or nitrogen atmosphere (b) optionally warming the mixing vessel to 70° C.; or (c) optionally allowing the temperature of the mixture to equilibrate under ambient conditions during the mixing process.

### EXAMPLES

### Example 1

Preparation of Polyorthoesters

The following syntheses illustrate the preparation of representative polyorthoesters. The starting materials are either commercially available or may be prepared as described in the preceding sections and in U.S. Pat. Nos. 4,549,010 and 5,968,543.

1(a) The polyorthoester in this example was prepared from 3,9-di(ethylidene)-2,4,8,10-tetraoxaspiro[5.5]undecane (DETOSU), triethylene glycol (TEG), and triethyleneglycol monoglycolide (TEG-mGL). The molar ratio of the three components (DETOSU:TEG:TEG-mGL) was 65:95:5.

Under rigorously anhydrous conditions, DETOSU (6.898 g, 32.5 mmol), TEG (7.133 g, 47.5 mmol) and TEG-mGL (0.521 g, 2.5 mmol) were weighed into a 250 mL round bottom flask, and the mixture dissolved in anhydrous ethyl acetate (16 mL). To this solution was added a salicylic acid solution in ethyl acetate (12 drops, 10 mg/mL) to initiate the polymerization. The solution came to a boil within a few minutes. The solution was allowed to cool to room temperature, then concentrated by rotoevaporation at 40-50° C. The flask was transferred to a vacuum oven, and dried at 40° C. for 2 hours followed by drying at 70° C. for additional 3 hours. The material was semi-solid with a molecular weight of about 4000.

1(b)The polyorthoester in this example was prepared from DETOSU, TEG, and tnethyleneglycol diglycolide (TEG-diGL). The molar ratio of the three components (DETOSU:TEG.TEG-diGL) was 65:80:20. Following the procedure of Example 1(a), DETOSU (6.898 g, 32.5 mmol), TEG (6.007 g, 40 mmol) and TEG-diGL (2.66 g, 10 mmol) were allowed to react. The reaction yielded a semi-solid material having a molecular weight of about 2000.

1(c) The polyorthoester in this example was prepared from DETOSU, TEG, and TEG-diGL. The molar ratio of the three components (DETOSU:TEG:TEG-diGL) was 60:70:30. Following the procedure of Example 1(a), DETOSU (25.47 g, 120 mmol), TEG (21.02 g, 140 mmol) and TEG-diGL (15.97 g, 60 mmol) were allowed to react. The reaction yielded a semi-solid material having a molecular weight of about 2000.

Other polyorthoesters, e.g. those containing diketene acetals of formulae V and VI and/or those containing other diols of formulae HO—R¹—OH, HO—R²—OH, HO—R³—OH, and HO—R⁴—OH, are prepared by similar methods.

1(d) The polyorthoester in this example was prepared from DETOSU, TEG and TEG-diGL. The molar ratio of the three components (DETOSU:TEG:TEG-diGL) was 90:80:20. Under rigorously anhydrous conditions, DETOSU (114.6 g, 540 mmol) was dissolved in a 2 L flask in 450 mL anhydrous THF and TEG (72.08 g, 480 mmol) and TEG-diGL (31.95 g, 120 mmol) was weighed into a 500 mL round bottom flask, and dissolved in anhydrous THF (50 mL). The TEG-diGL solution was added to the solution of DETOSU and TEG to initiate the polymerization. The solution came to a boil within a few minutes. The solution was allowed to cool to room temperature, then concentrated by rotary evaporation at 50° C., followed by rotary evaporation at 80° C. The material was semi-solid with a molecular weight of about 6,500.

### Example 2

Preparation of Pharmaceutical Compositions

Semi-solid pharmaceutical compositions with bupivacaine as the active agent were prepared by first milling the bupivacaine into fine particles and sieving, before mixing with selected amounts of a polyorthoester and an excipient. The mixing process was performed at room temperature under vacuum. Further size reduction of the bupivacaine particles was carried out by passing the semi-solid composition through a ball mill.

A. 60 wt. % polyorthoester (DETOSU/TEG/TEG-mGL 60:95:5)
40 wt. % bupivacaine. (control)

B. 40 wt. % polyorthoester (DETOSU/TEG/TEG-mGL 60:95:5)
40 wt. % bupivacaine
20 wt. % polyethylene glycol monomethyl ether 550.

C. 60 wt. % polyorthoester (DETOSU/TEG/TEG-diGL 60:80:20)
40 wt. % bupivacaine. (control)

D. 40 wt. % polyorthoester (DETOSU/TEG/TEG-diGL 60:80:20)
40 wt. % bupivacaine
20% wt. % polyethylene glycol monomethyl ether 550.

E. 20% wt. % polyorthoester (DETOSU/TEG/TEG-diGL 60:70:30)
40% wt. % bupivacaine
40% wt. % polyethylene glycol monomethyl ether.

Compositions B, D, and E had non-tacky, flowable texture. Compositions A and C had very sticky texture, were difficult to handle and showed poor syringability.

2(b) Semi-solid pharmaceutical compositions with mepivacaine as the active agent were prepared by dissolving the mepivacaine in the excipient ether 550 at a temperature between 120° C. and 150° C. in one vessel and mixing in the specified amount of the polyorthoester that was previously warned to 70° C. to make it allowable in a separate vessel. The formulation was additionally transferred once between the two vessels to ensure complete transfer of all components into a single vessel, and further mixed under an argon or nitrogen environment. This mixing may be carried out with or without warming the mixing vessel at 70° C. in order to maintain the flow characteristics necessary for a homogeneous distribution of all the components throughout the formulation. An example of a composition of such a formulation is shown below:
77.6 weight % polymer, such as a polyorthoester (molar ratio of DETOSU:TEG:TEG-diGL/90:80:20)
19.4 weight % polyethylene glycol monomethyl ether 550
3.0 weight % mepivacaine.

2(c) Semi-solid pharmaceutical compositions with granisetron as the active agent were prepared as described in Example 2(b) to obtain the following composition:
78.4 weight % polyorthoester (molar ratio of DETOSU:TEG:TEG-diGL/90:80:20)
19.6 weight % polyethylene glycol monomethyl ether 550
2.0 weight % granisetron.

2(d)A semi-solid delivery vehicle was prepared in a manner similar to that described in Example 2(b), with the omission of the step to dissolve the active pharmaceutical ingredient in the excipient. An example of a composition of a semi-solid delivery vehicle is shown below:
80 weight % polyorthoester (molar ratio of DETOSU:TEG:TEG-diGL/90:80:20)
20 weight % polyethylene glycol monomethyl ether 550.

2(e) Another polymer composition that is used in the product containing mepivacaine or granisetron comprises:
47.4 mole % DETOSU;
42.1 mole % triethylene glycol; and
10.5 mole % triethylene glycol glycolide.

In the above examples, the composition may also contain methoxy poly(ethylene glycol) [MPEG].

Other compositions containing other polyorthoesters, e.g. those containing diketene acetals of formula VI and those containing other diols of formulae HO—R¹—OH, HO—R²—OH, HO—R³—OH, and HO—R⁴—OH, and different active agents, and/or in different proportions are prepared in a similar manner.

### Example 3

Release Profiles of the Pharmaceutical Compositions:

The semi-solid compositions of Example 2 were weighed, placed into bottles with screw caps. 100 mL of 50 mM PBS (pH 7.4) was added to each bottle. The test bottles were transferred to a 37° C. incubator and placed on top of a rotor shaker (36 rpm). At various time points, bottles were removed from the incubator and samples of about 5 mL were removed and analyzed for bupivacaine content by HPLC at 263 nm. The remaining volume of buffer was removed and replaced with 100 mL fresh buffer.

Composition B had an increased rate of release over the control Composition A.

Composition D had a similar release rate as the control Composition C.

These test results demonstrated that the pharmaceutical compositions of the present invention have the advantage that the release rates of the composition may be adjusted and controlled in a variety of ways. The rates of release can be adjusted to accommodate a desired therapeutic effect by either altering the mole percentage of the α-hydroxyacid containing units in the polyorthoester as disclosed in U.S. Pat. No. 5,968,543, or by selecting a particular excipient, or by altering the concentration of the excipient in the composition, or the combination of all these factors.

The compositions can be irradiated, and the release rate of Composition E before and after irradiation showed no significant difference over twelve days using the test described above.

### Experimental Examples

1. Preparation of Poly(Ortho Ester)

Examples of two compositions of poly(ortho esters) prepared are described below:

(i) Polymer A:

The poly(ortho ester) in this example was prepared from DETOSU, TEG and TEG-diGL. The molar ratio of the three components (DETOSU:TEG:TEG-diGL) was 90::80::20. Under rigorously anhydrous conditions, DETOSU (114.61 g, 540 mmol) was dissolved in a 2 L flask in 450 mL anhydrous THF and TEG (72.08 g, 480 mmol) and TEG-diGL (31.95 g, 120 mmol) was weighed into a 500 mL round bottom flask, and dissolved in anhydrous THF (50 mL). The TEG-diGL solution was added to the solution of DETOSU and TEG to initiate the polymerization. The solution came to a boil within a few minutes. The solution was allowed to cool to room temperature, and then concentrated by rotary evaporation at 50° C. using a water aspirator, followed by rotary evaporation at 80° C. under high vacuum. The flask was then transferred to an oven at 70-75° C. and poured into an amber bottle over a period of about 30-60 minutes. The resulting polymer was semi-solid, viscous liquid at room temperature, with a weight average molecular weight (M_{w}) of approximately 6800 daltons.

(ii) Polymer B:

The poly(ortho ester) in this example was prepared from DETOSU and TEG, i.e., without any latent acid such as TEG-diGL. The molar ratio of the two components (DETOSU:TEG) was 90::100. The preparation method was identical to that described for Polymer A with the one difference. After the two components were mixed, approximately 0.05-1 mmol of salicylic acid was introduced dropwise, as a solution in THF (20 mg/mL), into the reaction mixture as a catalyst to initiate the reaction. The reaction was completed and worked-up in the same manner as Polymer A. The resulting polymer was a semi-solid, viscous liquid at room temperature, with a weight average molecular weight (M_{w}) of approximately 5300 daltons.

2. Preparation of Pharmaceutical Compositions:

(a) Semi-solid pharmaceutical compositions with mepivacaine free base as the active agent were prepared by dissolving the mepivacaine in the excipient polyethylene glycol monomethyl ether 550 (MPEG 550) at a temperature between 120° C. and 150° C. in one vessel and mixing in the specified amount of the poly(ortho ester) that was previously warmed to 70° C. to make it flowable in a separate vessel. The formulation was additionally transferred once between the two vessels to ensure complete transfer of all components into a single vessel, and further mixed under an argon or nitrogen environment. This mixing may be carried out with or without warming the mixing vessel at 70° C. in order to maintain the flow characteristics necessary for a homogeneous distribution of all the components throughout the formulation. Sterilization of the formulation was carried out by gamma-irradiation at 22-32 kilograys.

The following compositions containing mepivacaine were prepared:

(i) Formulation A:

79.2 weight % Polymer A (molar ratio of DETOSU:TEG:TEG-diGL/90::80::20)

19.8 weight % polyethylene glycol monomethyl ether 550

1.0 weight % mepivacaine free base

(ii) Formulation B:

78.4 weight % Polymer A (molar ratio of DETOSU.TEG:TEG-diGL/90::80::20)

19.6 weight % polyethylene glycol monomethyl ether 550

2.0 weight % mepivacaine free base

(iii) Formulation C:

77.6 weight % Polymer A (molar ratio of DETOSU:TEG:TEG-diGL/90::80::20)

19.4 weight % polyethylene glycol monomethyl ether 550

3.0 weight % mepivacaine free base

(iv) Formulation D:

77.6 weight % Polymer B (molar ratio of DETOSU:TEG/90::100)

19.4 weight % polyethylene glycol monomethyl ether 550

3.0 weight % mepivacaine free base

(b) Semi-solid pharmaceutical compositions with granisetron free base as the active agent were prepared as described in Example 2(a) to obtain the following compositions:

(i) Formulation E:

79.2 weight % Polymer A (molar ratio of DETOSU:TEG:TEG-diGL/90::80::20)

19.8 weight % polyethylene glycol monomethyl ether 550

1.0 weight % granisetron free base

(ii) Formulation F:

78.4 weight % Polymer A (molar ratio of DETOSU:TEG:TEG-diGL/90::80::20)

19.6 weight % polyethylene glycol monomethyl ether 550

2.0 weight % granisetron free base

(iii) Formulation G:

77.6 weight % Polymer A (molar ratio of DETOSU:TEG:TEG-diGL/90::80::20)

19.4 weight % polyethylene glycol monomethyl ether 550

3.0 weight % granisetron free base

(c) Semi-solid pharmaceutical compositions including both granisetron and mepivacaine were prepared as described in Example 2(a) to obtain the following compositions:

(i) Formulation H:

77.6 weight % Polymer A (molar ratio of DETOSU:TEG:TEG-diGL/90::80:20)

19.4 weight % polyethylene glycol monomethyl ether 550

1.0 weight % granisetron free base

2.0 weight % mepivacaine free base

(ii) Formulation I:

76.8 weight % Polymer A (molar ratio of DETOSU:TEG:TEG-diGL/90::80::20)

19.2 weight % polyethylene glycol monomethyl ether 550

1.0 weight % granisetron free base

3.0 weight % mepivacaine free base

(d) Placebo semi-solid delivery vehicles were prepared in a manner similar to that described in Example 2(a), with the omission of the step to dissolve the active pharmaceutical ingredient in the excipient. Two examples of compositions of semi-solid delivery vehicles based on poly(ortho esters) with and without latent acid are shown below:

(i) Formulation J:

80 weight % Polymer A (molar ratio of DETOSU:TEG:TEG-diGL/90:80::20)

20 weight % polyethylene glycol monomethyl ether 550

(ii) Formulation K:

80 weight % Polymer B (molar ratio of DETOSU:TEG/90::100)

20 weight % polyethylene glycol monomethyl ether 550

(e) Semi-solid pharmaceutical compositions including buprenorphine free base were prepared in manner described in Example 2(a) to obtain the following compositions:

(i) Formulation L

79.2 weight % Polymer A (molar ratio of DETOSU:TEG:TEG-diGL/90::80::20)

19.8 weight % polyethylene glycol monomethyl ether 550

1.0 weight % buprenorphine free base

(ii) Formulation M

77.6 weight % Polymer A (molar ratio of DETOSU:TEG:TEG-diGL/90::80::20)

19.4 weight % polyethylene glycol monomethyl ether 550

2.0 weight % buprenorphine free base

3. In Vitro Release of Active From Semi-Solid Pharmaceutical Compositions

50-60 mg of gamma-irradiated semi-solid pharmaceutical compositions prepared as described in examples 2(a) and 2(b) were transferred into tared and labeled scintillation vials (called Main Vials) in triplicate, and their masses recorded to 0.0001 g. The vials were tared again and 20 g of PBS (phosphate buffered saline) transferred into each of them. The masses of the added PBS were recorded to 0.01 g. The vials were then securely sealed with PTFE-faced rubber lined caps, documenting any observations in the laboratory notebook, and then placed in a 37° C. incubator, noting the time and temperature. Analytical samples were taken immediately before capping and at 1, 2, 4, 6, and 24 hours and then at 24-hour intervals, until all active was released (exceptions to the sampling schedule were made for weekends and holidays). Again, all masses were recorded to 0.0001 g. The samples were removed by giving each vial a gentle swirl to mix the solution, without shaking violently. The sample ID, time, and presence/absence of solids were recorded in the lab notebook. The solids, if any, were allowed to settle for 5 minutes, before the vial was opened to remove a 10 g aliquot by pipette. The aliquot was filtered through a 0.45 μm syringe filter into a separate, labeled vial (Analytical Vial). The volume of withdrawn buffer was replaced with 10 g fresh buffer into the tared Main Vial, which was then re-capped and replaced into the incubator. 1.5 mL of the solution from the Analytical Vial was placed into an appropriate, labeled HPLC vial for quantitative HPLC analysis of the active released into the buffer at each time point.

The following information was used to generate a profile of the cumulative release of the weight-percent of the active over time:
concentration of active obtained from HPLC analysis of pulled aliquots
recorded weights of depots at the beginning of the experiment
weights and times of both, the pulled aliquots (for HPLC analysis) and the replaced (fresh) buffer.

In addition, regression parameters were calculated for the initial, linear portion of the release curve, based on which the release of the active was quantified at interpolated times of 4 hours and 20 hours for formulations with mepivacaine and granisetron respectively.

(a) Comparison of in vitro release of mepivacaine from formulations containing different levels of mepivacaine is shown below. The in vitro release of mepivacaine from formulations containing two different levels of mepivacaine is seen in the following graph:

[image]

This experiment illustrates some effects of the incremental addition of mepivacaine to the vehicle Formulation J. The in vitro release data shows that both formulations release mepivacaine at the same rate over 24 hours, but then Formulation A (1 wt % mepivacaine) has its main release to achieve 100% by ˜72 hours (3 days). This is in contrast with Formulation B (2 wt % mepivacaine) and Formulation C (3 wt % mepivacaine), where the release of mepivacaine extends to between 110 hours and 160 hours (4-6 days).

(b) Comparison of in vitro release of granisetron from formulations containing different levels of granisetron is shown below. The in vitro release of granisetron from three formulations containing two different levels of granisetron is seen in the following graph:

[image]

The in vitro release profiles showed typical profiles for Formulation J (vehicle) combined with granisetron. The most important feature of the composite profile is that the formulation with 2 wt % granisetron (Formulation F) had a longer release, whereas the 1 wt % formulation (Formulation E) showed a more rapid release. This indicates that 2 wt % granisetron extends the release of granisetron out past 1 wt %. The addition of another 1 wt % granisetron (Formulation G, 3 wt %) does extend the release profile further. These observations indicate a role for the basic active pharmaceutical ingredient in the kinetics of its in vitro release from the formulation.

(c) Comparison of in vitro release of granisetron from formulations with and without added mepivacaine is shown below. The in vitro release of granisetron from formulations with 1 wt % granisetron with and without added mepivacaine is seen in the following graph:

[image]

The results of this experiment illustrate the effect of adding a different base stabilizer (mepivacaine) in extending the release of the active ingredient of interest (granisetron). Thus, both formulations studied in this experiment contained 1 wt % granisetron. However, the addition of mepivacaine had a marked impact in extending the release of 1 wt % granisetron from 72-96 hours (3-4 days) for the formulation without additional mepivacaine (Formulation E) to around 160 hours (6 days) for the formulations with added 2 and 3 wt % mepivacaine (Formulation H).

(d) In vitro release of mepivacaine from formulations based on Polymer A (with latent acid) and Polymer B (without latent acid) is shown below. The in vitro release of mepivacaine from Formulation C (consisting of POE with latent acid) and Formulation D (consisting of POE without latent acid) is seen in the following graph:

The data indicates that the release profile for both formulations is quite similar up to the 4^{th} day. The next data point at the 6^{th} day shows the divergence between the two formulations, as seen by the more gradual release continuing through Day 16 for Formulation D, versus complete release seen by Day 6 for Formulation C. This data, in conjunction with the visual erosion data presented in Example 4 (d), shows the relative extension of the erosion and hence release of the active upon the addition of a base, irrespective of the inclusion of latent acid in the component poly(ortho ester).

4. Visual Monitoring of Erosion of Semi-Solid Pharmaceutical Compositions

This procedure was carried out similarly to that for the in vitro release experiment described in Example 3, with one difference: the buffer was not changed or replaced for the duration of the experiments. Photographs were taken at different time points to record the appearance of the depot in the buffer over time, under conditions similar to those for the in vitro release experiments, i.e., 37° C., pH 7.4.

(a) Comparison of erosion of Formulation J (0 wt % mepivacaine) and Formulation C (3 wt % mepivacaine):

Photographic documentation of a side-by-side comparison of the erosion of Formulation J and Formulation C under conditions for in vitro release (PBS, pH 7.4, 37° C.) showed the following: At the initial time point all samples were visually present. At the I hour time point, the samples of Formulation C did not exhibit any significant differences, while the size of the samples from Formulation J appeared to be less than half the size of the original aliquot. At 2 hours, there was no significant difference in the appearance of samples from Formulation C, while, for Formulation J, there was a minute amount of sample left—there were a few specks of formulation left which were seen in small round bubbles stuck to the bottom and walls of the scintillation vial. By 6 hours, all of the Formulation J specks disappeared. At the same time point for Formulation C, there were still no significant differences being observed. For Formulation C there was no significant difference for the 6 hour and 1 day time points. At 2 days the samples became smaller. At 3 days they were even smaller and by the 6th day the samples completely disappeared. Thus, in this experiment, Formulation J completely eroded between 3-6 hours while Formulation C completely eroded in 4 to 6 days. It was concluded that Formulation J erodes more rapidly than Formulation C.

(b) Comparison of erosion of Formulation J (0% granisetron) and Formulation F (2% granisetron):

Photographic documentation of a side-by-side comparison of the erosion of Formulation J and Formulation F under conditions for in vitro release showed the following: At the initial time point all samples were visually present. At 2 hours, the Formulation F samples did not exhibit any significant differences, while the size of the samples from Formulation J appeared to be less than half the size of the original aliquot. At 4 hours, there was no significant difference in the appearance of samples from Formulation F. In the samples of Formulation J there was a minute amount of sample left—there were a few specks of polymer left distributed in small round bubbles stuck to the bottom and walls of the scintillation vial. By 6 hours, all of the Formulation J specks disappeared. For Formulation F, there were still no significant differences observed. For Formulation F, there were no significant differences between the 6 hour, 1 day and 3 day time points. At 6 days, the samples became visually much smaller. At 7 and 10 days they were getting progressively smaller and by the 13th day the samples completely disappeared. In this experiment, Formulation J completely eroded between 3-6 hours while Formulation F completely eroded in 10 to 13 days. It was concluded that Formulation J erodes more rapidly than Formulation F.

(c) Comparison of erosion of Formulation J (0% mepivacaine), Formulation A (1% mepivacaine), Formulation B (2% mepivacaine) and Formulation C (3% mepivacaine):

Photographic documentation of the side-by-side erosion of semi-solid depots containing 0%, 1%, 2% and 3% mepivacaine under conditions for in vitro release showed that that Formulation J (0% mepivacaine) eroded by 6 hours, Formulation A (1% mepivacaine) eroded by 7 days, Formulation B (2% mepivacaine) eroded by 7 days, and Formulation C (3% mepivacaine) eroded by approximately 14 days. This experiment illustrates that the addition of mepivacaine to Formulation J has a profound effect on the formulation in aqueous systems as observed by the persistence of the depot in an erosion study. It was observed that the persistence of the depot dramatically increased by the addition of even 1% mepivacaine (Formulation A), while the addition of more mepivacaine (2 and 3 wt %; Formulation B and Formulation C) increased this persistence.

(d) Comparison of erosion of formulations based on Polymer A (with latent acid) and Polymer B (without latent acid)—Formulation J (based on Polymer A—with latent acid, 0% mepivacaine), Formulation D (based on Polymer B—without latent acid, 0% mepivacaine), Formulation C (based on Polymer A—with latent acid, 3% mepivacaine) and Formnulation K (based on Polymer B—without latent acid, 3% mepivacaine): Photographic documentation of the side-by-side erosion of semi-solid depots containing 0% mepivacaine and 3% mepivacaine based on Polymers A and B with and without latent acid respectively, under conditions for in vitro release, showed that two formulations without mepivacaine eroded very quickly (in approximately 1 day). Comparing the formulation pairs with and without mepivacaine, it was seen that in both types of formulations (based on polymers with and without latent acid), the addition of mepivacaine retarded the erosion significantly. Thus, Formulation C eroded completely by Day 8, while 95% of Formulation K had eroded by approximately 35 days and completely eroded by Day 46. It was concluded that in the absence of a base such as mepivacaine or granisetron, there was no effect of the presence or absence of latent acid on the erosion of semi-solid poly(ortho ester) formulations. Moreover, both types of formulations (based on polymers with and without latent acid) took longer to erode in the presence of 3% mepivacaine. The erosion of the formulation was longer for Formulation K, which was based on Polymer B, without latent acid.

Demonstration of Stabilization of Formulations with a Wide Range of MPEG 550 Content:

2. Preparation of Pharmaceutical Compositions:

(e) Semi-solid formulation compositions based on Polymer A with 2 weight % granisetron free base and increasing amounts of MPEG 550 were prepared as per the following table:

| | | | MPEG 550 as a | |
|---|---|---|---|---|
| | | | percent of | |
| | | | Polymer A + | Granisetron |
| | Polymer A | MPEG 550 | MPEG 550 | free base |
| Formulation | (weight %) | (weight %) | (weight %) | (weight %) |
| Formulation F1 | 93.1% | 4.9% | 5% | 2.0% |
| Formulation F2 | 88.2% | 9.8% | 10% | 2.0% |
| Formulation F | 78.4% | 19.6% | 20% | 2.0% |
| Formulation F3 | 68.6% | 29.4% | 30% | 2.0% |
| Formulation F4 | 58.8% | 39.2% | 40% | 2.0% |
| Formulation F5 | 49.0% | 49.0% | 50% | 2.0% |
| Formulation F6 | 39.2% | 58.8% | 60% | 2.0% |
| Formulation F7 | 29.4% | 68.6% | 70% | 2.0% |
| Formulation F8 | 19.6% | 78.4% | 80% | 2.0% |
| Formulation F9 | 9.8% | 88.2% | 90% | 2.0% |
| Formulation F10 | 0% | 98.0% | 100% | 2.0% |

3. In Vitro Release of Active From Semi-Solid Pharmaceutical Compositions

(f) In vitro release of granisetron from formulations based on Polymer A and containing increasing amounts of MPEG 550:

The in vitro release of granisetron from Formulations F-F10, described in Example 2(e), is shown in the following graphs;

[image]

The data shows that after the first 24 hours, the rate of release of granisetron is reduced (extended) as the MPEG 550 content (as a percentage of Polymer A and MPEG 550 content) is increased from 5 weight % to 20 weight %. Further increases in the MPEG 550 content in increments of 10 weight % are clearly associated with a corresponding increase in the amount of granisetron released at each time point, i.e. an increase in the rate of release. This continues until 80 weight % MPEG 550, with the two compositions containing 90 weight % and 100 weight % MPEG 550 being essentially indistinguishable from each other all the granisetron is released from these two formulations almost immediately without any apparent control. Thus, the above data shows that for this set of components (Polymer A, MPEG 550, granisetron), some degree of control is achieved in release of the active pharmaceutical ingredient when the formulation contains as little as approximately 20 weight % poly(ortho ester) and 2 weight % of a basic excipient. In this example, this control is gradually increased (with the rate of granisetron release progressively reduced) as the polymer content is increased in the formulations while keeping the basic excipient at 2 weight %.

As the data shows, the composition with 20% MPEG 550, exemplified by Formulation F, has the slowest rate of granisetron release, the composition of which may be particularly useful for clinical trials. In addition, varying degrees of control are achievable as desired by varying the MPEG 550 content in the composition.

Representative data for the release rate for animal and human trials demonstrating the application of the polymer formulations of the present invention are provided below:

A. Rat study 24-06, test article Formulation C IVR data:

[image]

B. Rat study 24-06, test article Formulation C pharmacokinetics data:

[image]

C. Clinical study C2003-01, test article Formulation C pharmacokinetics data:

[image]

D. Rat study 25-06 (Buprenorphine), test article pharmacokinctics data:

[image]

E. Clinical study C2004-01, healthy normals, test article pharmacokinetics data:

[image]

F. Clinical study C2005-01, chemotherapy patients, test article pharmacokinetics data:

[image]

G. Clinical studies C2004-01 and C2005-01, healthy normals and chemotherapy patients, pharmacokinetics comparison:

[image]

The foregoing is offered primarily for purposes of illustration. It will be readily apparent to those skilled in the art that the molecular structures, proportions of the various components in the delivery vehicle or pharmaceutical composition, method of manufacture and other parameters of the invention described herein may be further modified or substituted in various ways without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A pharmaceutical composition comprising:
(A) a semi-solid delivery vehicle, comprising
a polyorthoester of formula I, 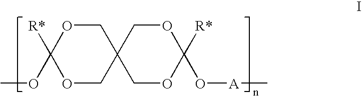
where:
R* is a C₁₋₄ alkyl;
n is an integer of at least 5; and
A is R¹, R³, or R⁴, where
R¹ is: 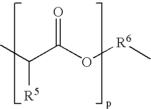
where:
p is an integer of 1 to 20;
R³ and R⁶ are each independently: 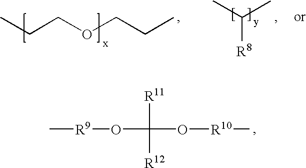
where:
x is an integer of 0 to 30;
y is an integer of 2 to 200;
R⁸ is hydrogen or C₁₋₄ alkyl;
R⁹ and R¹⁰ are independently C₁₋₁₂ alkylene;
R¹¹ is hydrogen or C₁₋₆ alkyl and R¹² is C₁₋₆ alkyl; or R¹¹ and R¹² together are C₃₋₁₀ alkylene;
R⁴ is a diol containing at least one functional group independently selected from amide, imide, urea, and urethane groups; and
R⁵ is hydrogen or C₁₋₄ alkyl;
in which at least 0.01 mol percent of the A units are of the formula R¹, and wherein the polyorthoester has a lifetime of 12 hours or less in vitro;
(B) a pharmaceutically acceptable, polyorthoester-compatible liquid excipient selected from polyethylene glycol ether derivatives having a molecular weight between 200 and 4000, polyethylene glycol copolymers having a molecular weight between 200 and 10,000, mono-, di- or tri-glycerides of a C₂₋₁₉ aliphatic carboxylic acid or a mixture of such acids, alkoxylated tetrahydrofurfuryl alcohols and their C₁₋₄ alkyl ethers and C₂₋₁₉ aliphatic carboxylic acid esters, and biocompatible oils; and
(C) a polyorthoester stabilizing agent that extends the lifetime of the polyorthoester by at least one lifetime, wherein said stabilizing agent is a basic biologically active agent.

2. The pharmaceutical composition of claim 1, wherein the stabilizing agent is an amine comprising biologically active organic compound or its salt.

3. The pharmaceutical composition of claim 2, wherein the biologically active organic compound is selected from the group consisting of anti-infectives, antiseptics, steroids, therapeutic polypeptides, anti-inflammatory agents, cancer chemotherapeutic agents, narcotics, local anesthetics, antiangiogenic agents, vaccines, antigens, DNA, RNA and antisense oligonucleotides.

4. The pharmaceutical composition of claim 3, wherein the biologically active organic compound is a local anesthetic.

5. The pharmaceutical composition of claim 4, wherein the local anesthetic is selected from the group consisting of bupivacaine, lidocaine, mepivacaine, pyrrocaine and prilocaine.

6. The pharmaceutical composition of claim 3, wherein the biologically active organic compound is a cancer chemotherapeutic agent.

7. A method of stabilizing a polyorthoester polymer having a lifetime of 12 hours or less in vitro, the method comprising adding to the polymer a stabilizing agent wherein said stabilizing agent is a basic biologically active agent and the stabilizing agent extends the lifetime of the polymer by at least one lifetime or at least two lifetimes.

8. The method of claim 7, wherein the stabilizing agent is an amine comprising a biologically active organic compound or its salt.
